SEQUENCE LISTING (28 sequences)

SEQ ID NO: 1 - protein, 120 aa, Synthetic construct [sequence omitted]

SEQ ID NO: 2 - protein, 107 aa, Synthetic construct [sequence omitted]

SEQ ID NO: 3 - protein, 8 aa, Mus musculus
GYTFISYW

SEQ ID NO: 4 - protein, 8 aa, Mus musculus
IDPDSGGT

SEQ ID NO: 5 - protein, 11 aa, Mus musculus
AREGDYAWFAY

SEQ ID NO: 6 - protein, 11 aa, Mus musculus
QSIVHSNGNTY

SEQ ID NO: 7 - skipped

SEQ ID NO: 8 - protein, 9 aa, Mus musculus
FQGSHVPWT

SEQ ID NO: 9 - protein, 8 aa, Mus musculus
GFTFNIYA

SEQ ID NO: 10 - protein, 10 aa, Mus musculus
IRSKSNNYAT

SEQ ID NO: 11 - protein, 12 aa, Mus musculus
VSSYYSGSFFAY

SEQ ID NO: 12 - protein, 13 aa, Mus musculus
SQSIVYSNGKIYL

SEQ ID NO: 13 - skipped

SEQ ID NO: 14 - protein, 9 aa, Mus musculus
FQGSHLPLT

SEQ ID NO: 15 - protein, 118 aa, Mus musculus
QVQLQQPGAALVKPGASVKLSCKASGYTFISYWMLWVKQRPGRGLEWIGRIDPDSGGTKYNEKFKSKATLTVDKSSSTAYMQLSSLTSEDSAVYYCAREGDYAWFAYWGQGTLVPVSA

SEQ ID NO: 16 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 17 - protein, 121 aa, Mus musculus
EVQLVESGGGLVQPKGSLKLSCAASGFTFNIYAMNWIRQAPGKGLEWIARIRSKSNNYATYYADSVKDRFTISRDDSQNMVYLQMNNLKTEDTAMYYCVSSYYSGSFFAYWGQGTLVTVSA

SEQ ID NO: 18 - protein, 113 aa, Mus musculus
DVLMTQTPLSLPVSLGDQASISCRSSQSIVYSNGKIYLEWYLQKPGQSPKLLIYKVSNRFSGVPDRFSGSGSGTDFTLKISRVEAEDLGVYYCFQGSHLPLTFGAGTKLELKR

SEQ ID NO: 19 - protein, 225 aa, Synthetic construct
QVQLQQPGAALVKPGASVKLSCKASGYTFISYWMLWVKQRPGRGLEWIGRIDPDSGGTKYNEKFKSKATLTVDKSSSTAYMQLSSLTSEDSAVYYCAREGDYAWFAYWGQGTLVTVSCASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 20 - protein, 219 aa, Synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 228 aa, Synthetic construct
EVQLVESGGGLVQPKGSLKLSCAASGFTFNIYAMNWIRQAPGKGLEWIARIRSKSNNYATYYADSVKDRFTISRDDSQNMVYLQMNNLKTEDTAMYYCVSSYYSGSFFAYWGQGTLVTVSCASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 22 - protein, 219 aa, Synthetic construct
DVLMTQTPLSLPVSLGDQASISCRSSQSIVYSNGKIYLEWYLQKPGQSPKLLIYKVSNRFSGVPDRFSGSGSGTDFTLKISRVEAEDLGVYYCFQGSHLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 20 aa, Synthetic construct
GGGGSGGGGSGGGGSGGGGS

SEQ ID NO: 24 - DNA, 13 bp, Synthetic construct
  source 1..13: mol_type = other DNA
gccgccrccatgg

SEQ ID NO: 25 - protein, 250 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIKGGGGSGGGGSGGGGSGGGGSQVQLQQPGAALVKPGASVKLSCKASGYTFISYWMLWVKQRPGRGLEWIGRIDPDSGGTKYNEKFKSKATLTVDKSSSTAYMQLSSLTSEDSAVYYCAREGDYAWFAYWGQGTLVTVSC

SEQ ID NO: 26 - protein, 253 aa, synthetic construct
DVLMTQTPLSLPVSLGDQASISCRSSQSIVYSNGKIYLEWYLQKPGQSPKLLIYKVSNRFSGVPDRFSGSGSGTDFTLKISRVEAEDLGVYYCFQGSHLPLTFGAGTKLELKGGGGSGGGGSGGGGSGGGGSEVQLVESGGGLVQPKGSLKLSCAASGFTFNIYAMNWIRQAPGKGLEWIARIRSKSNNYATYYADSVKDRFTISRDDSQNMVYLQMNNLKTEDTAMYYCVSSYYSGSFFAYWGQGTLVTVSC

SEQ ID NO: 27 - protein, 118 aa, Synthetic construct
QVQLQQPGAALVKPGASVKLSCKASGYTFISYWMLWVKQRPGRGLEWIGRIDPDSGGTKYNEKFKSKATLTVDKSSSTAYMQLSSLTSEDSAVYYCAREGDYAWFAYWGQGTLVTVSC

SEQ ID NO: 28 - protein, 121 aa, Synthetic construct
EVQLVESGGGLVQPKGSLKLSCAASGFTFNIYAMNWIRQAPGKGLEWIARIRSKSNNYATYYADSVKDRFTISRDDSQNMVYLQMNNLKTEDTAMYYCVSSYYSGSFFAYWGQGTLVTVSC

(19) United States
(12) Patent Application Publication
(10) Pub. No.: US 2026/0265367 A1
(43) Pub. Date: Sep. 10, 2026

(54) MUTANT FAB FRAGMENT FOR OBTAINING SITE-SPECIFIC MONO-OR BIFUNCTIONALISED CONJUGATES

(71) Applicants: COMMISSARIAT A L'ENERGIE ATOMIQUE ET AUX ENERGIES ALTERNATIVES, Paris (FR); UNIVERSITE DE BOURGOGNE, Dijon (FR); CENTRE NATIONAL DE LA RECHERCHE SCIENTIFIQUE (CNRS), Paris (FR)

(72) Inventors: Didier BOQUET, Gif-sur-Yvette cedex (FR); Amaury HERBET, Gif-sur-Yvette cedex (FR); Narciso COSTA, Gif-sur-Yvette cedex (FR); Marie HAUTIERE, Gif-sur-Yvette cedex (FR); Franck DENAT, Dijon (FR); Claire BERNHARD, Dijon (FR); Victor GONCALVES, Dijon (FR); Delphine VIVIER, Dijon (FR)

(21) Appl. No.: 19/154,049

(22) PCT Filed: Feb. 9, 2024
(86) PCT No.: PCT/FR2024/050178

§ 371 Date: Aug. 6, 2025

(30) Foreign Application Priority Data
Feb. 9, 2023 (FR) FR2301200

(51) Int. Cl. C07K 16/26; A61K 49/00; A61K 51/10

(52) U.S. Cl. CPC C07K 16/26; A61K 49/0032; A61K 49/0058; A61K 51/1036; A61K 51/1093; A61K 2123/00; C07K 2317/24; C07K 2317/55; C07K 2317/565; C07K 2317/92

(57) ABSTRACT

The present invention relates to an antigen-binding mutant antibody fragment comprising a mutated heavy chain variable domain, wherein the amino acid residue at position 128 according to the IMGT nomenclature of the heavy chain variable domain is substituted by a cysteine, provided that this fragment is not a Fab fragment in which the mutated heavy chain fragment corresponds to the amino acid sequence SEQ ID NO: 1 and the light chain fragment corresponds to the amino acid sequence SEQ ID NO: 2. The present invention also relates to nucleic acid molecules, vectors and host cells that can be used for the production of the mutant antibody fragment, final and intermediate conjugates comprising same and methods for the production of same, as well as various uses thereof.

### FIELD OF THE INVENTION

The present invention relates to a mutant Fab fragment, characterised in that the amino acid residue in position 128 of the heavy chain according to the IMGT nomenclature is substituted by a cysteine, provided that said fragment is not a Fab fragment in which the mutated heavy chain fragment corresponds to the amino acid sequence SEQ ID NO: 1 and the light chain fragment corresponds to the amino acid sequence SEQ ID NO: 2, as well as its use for obtaining site-specific mono-functionalised or bi-functionalised conjugates thereof, an intermediate conjugate, methods for preparing the final or intermediate conjugate and the mono-functionalised or bi-functionalised conjugates, as well as various uses of these conjugates.

### PRIOR ART

The use of isotopic imaging agents in which an imaging radioisotope (positron emitter or gamma emitter) is attached to a vector specific to receptors over-expressed by tumour cells is now a highly effective approach for diagnosing many cancers. Furthermore, the recent clinical successes of Lutathera and PSMA-617, and the earlier example of Zevalin, confirm the efficacy of such radiopharmaceuticals for therapy (respectively of neuroendocrine tumours, prostate cancer and non-Hodgkin's lymphoma) when the radioisotope is a beta emitter (e.g. Lu-177 or Y-90) or alpha emitter (e.g. Ac-225 or Pb-212).

Moreover, there is growing interest in the use of fluorescent conjugates to assist surgery (Fluorescence Guided Surgery, FGS). The presence of a fluorescent molecule helps the surgeon to view tumour tissue that is invisible to the naked eye, enabling better delineation of tumour areas and therefore better tumour resection, thus reducing the risk of recurrence.

Developing these approaches involves modifying the biological vector in order to introduce the desired functionality (cytotoxic molecule, chelating agent for a radiometal, fluorophore, etc.). Numerous bioconjugation methods have been developed, including some so-called “site-specific” approaches for controlling the binding site on proteins. However, there are few examples of strategies that are sufficiently modular to allow several functions to be introduced in the same way.

A few examples of conjugates containing both a radiometal chelating agent and a fluorophore have been described, in particular in the case of vectors based on a whole antibody, but these are often constructed using sequential functionalisation methods that do not allow precise control of the ratio of the two imaging probes and good reproducibility of the syntheses. For example, a trastuzumab conjugated to a tetrazine platform bifunctionalised by a chelating agent (DOTAGA) and a fluorophore (disulfonated Cy5) has been described, but the grafting of the bifunctionalised tetrazine platform was carried out in a random and non-site-specific manner, by reaction of a bicyclo[6.1.0]nonyne (BCN) group on the amine functions of the antibody (WO2018172543A1 and Coline Canovas, et al. Modular Assembly of Multimodal Imaging Agents through an Inverse Electron Demand DieLs-Alder Reaction. Bioconjugate Chemistry, American Chemical Society, 2019, 30(3), pp. 888-897). Similarly, a diabody modified by the addition of a C-terminal H₆-GGC tag (6 histidines followed by two glycines and one cysteine) and conjugated to these two cysteines by a fluorophore (IRDye800CW) and randomly by a radioisotope (¹²⁴lodine) has been synthesised (Zettlitz, K. A., et al., 2018. Dual-modality immunoPET and near-infrared fluorescence (NIRF) imaging of pancreatic cancer using an anti-prostate cancer stem cell antigen (PSCA) cys-diabody. J Nucl Med jnumed.117.207332). However, random grafting does not guarantee the maintenance of affinity for the antigen, nor does it ensure good reproducibility of the conjugate obtained with each batch produced, which is of course unacceptable for conjugates intended to be administered to patients.

A trastuzumab bifunctionalised in a site-specific manner by a chelating agent (DFO) and a fluorophore (IRDye800CW) has been described, the conjugate being based on a lysine platform trifunctionalised by DFO, IRDye800CW and a BCN group, and its grafting onto the antibody at the two N-glycans, previously modified to comprise an azide group reacting with the BCN group of the lysine platform (Pierre Adumeau, et al. Site-Specific Platform-Based Conjugation Strategy for the Synthesis of Dual-Labeled Immunoconjugates for Bi-modal PET/NIRF Imaging of HER2-Positive Tumors. Bioconjugate Chemistry, American Chemical Society, 2022, 33(3), pp. 530-540).

However, in the case of brain tumours, the conjugates must also be able to cross the blood-brain barrier (BBB). Conjugates based on a whole antibody have difficulty crossing the BBB and are therefore not well suited to brain tumours. In addition, whole antibodies have relatively long in vivo lifespans (in particular IgG) due to their Fc domain, which is undesirable, in particular for diagnostic purposes.

These two problems of a highly selective barrier and in vivo lifespan could be overcome using smaller antibody formats. A conjugate was synthesised between an anti-EGFR VHH, a photosensitising fluorophore (IRDye700DX) and a chelating agent (diethylenetriamine pentaacetic acid or DTPA) via a tetrazine platform. The VHH was grafted onto the tetrazine platform by grafting onto a cysteine added to the C-terminus of the VHH (Renard, E. et al. Site-Specific Dual-Labeling of a VHH with a Chelator and a Photosensitiser for Nuclear Imaging and Targeted Photodynamic Therapy of EGFR-Positive Tumors. Cancers 2021, 13, 428).

WDO2011/003622A1 describes a bivalent anti-HER2 nanobody comprising two VH domains fused via a linker (Nanobody 1-(VVTS)C of sequence SEQ ID NO: 17). In the 2^{nd} VH domain of the nanobody, the amino acid at IMGT position 128 has been replaced by a cysteine. Although the nanobody has been produced, its ability to bind its antigen with the same affinity as without the substitution at IMGT position 128 was not verified. Moreover, no conjugate of this nanobody with another chemical group at the thiol group of the cysteine inserted at IMGT position 128 has been described.

WO2011/006914A2 describes a variant called DSM0162 of an anti-TNFR1 single-domain antibody (called DOM1 h-574-16), in which the amino acid at IMGT position 128 has been substituted by a cysteine, as well as a conjugate of this fragment to polyethylene glycol (PEG) via a maleimide-type linker, with the aim of increasing its half-life in vivo. A half-life was calculated on the basis of an ELISA test using capture by TNFR1. However, no coupling efficiency is indicated and it has not been demonstrated that the affinity for the antigen is unaffected by the substitution.

US2006/062784A1 describes a variant (called DOM8-24cys of sequence SEQ ID NO: 85) of an anti-CD40 single domain antibody (DOM-24, SEQ ID NO: 26), in which the amino acid at IMGT position 128 has been substituted by a cysteine, as well as the possibility of conjugating it to polyethylene glycol (PEG). However, no precise conjugation procedure is described, no coupling efficiency is mentioned and no data confirming that the variant and the conjugate maintain the same affinity for their CD40 antigen are presented.

WO2018/106895A1 describes two fusion proteins of sequences SEQ ID NO: 816 and 817 (Table 4 of Example 2) comprising the Shiga toxin subunit A fused to a VHH of unknown antigenic specificity, in which the amino acid at IMGT position 128 has been substituted by a cysteine.

However, the activity of these two fusion proteins has not been tested, and so they have not been shown to maintain their affinity for their antigen. Moreover, no conjugate of these specific fusions is described.

WO2022/133089A1 describes a single-chain antibody (sdAb) directed against CEA6 called Cap03-04-CEA6-R3-39/19 of sequence SEQ ID NO: 154, in which the amino acid at IMGT position 128 has been substituted by a cysteine. However, no functional data confirming that this sdAB maintains equivalent affinity for its antigen is presented. No conjugate of this specific sdAb is described either.

However, VHHs are not necessarily available for all targets of interest, whereas many antibodies against targets of interest are available. The use of a small antibody fragment, such as a Fab or scFv fragment (or any other antibody fragment format with a heavy chain and a light chain), could therefore make it possible to benefit from all the existing antibodies aimed at targets of interest, while limiting the size sufficiently to allow crossing of the BBB, and limiting the in vivo lifespan, due to the absence of an Fc domain.

WO2010/115866A1 describes an anti-GPIIb/IIIa scFv (AP3 LC-HC scFV C39S S248C). In the part of scFv corresponding to the VH domain, the amino acid at IMGT position 128 has been substituted by a cysteine. Conjugates of scFv with a chemical group comprising polyethylene glycol (PEG) (Example 13) or a variant of FVIII (Example 14), the conjugation involving the thiol group of cysteine, are also described. However, no coupling efficiency is indicated. Moreover, this document does not present any data confirming that the conjugates are functional and retain their affinity for the GPIIb/IIIa antigen. On the contrary, in both cases, the data from the crude analysis of the conjugates suggests that the material obtained contains a large proportion of aggregated material, unlikely to retain its affinity for the antigen.

A Fab fragment would be particularly suitable, given its intermediate molecular weight (neither too high nor too low), enabling it to cross the BBB and have an in vivo lifespan that is neither too long nor too short. Moreover, the presence of two partial heavy chains may allow double conjugation, which would be absent in fragments of the VHH or ScFv type, resulting in improved specific activity for both fluorescence and isotopic imaging.

However, there are difficulties in preparing bioconjugates based on a Fab fragment. In particular, a Fab fragment generally does not comprise an N-glycan that can be modified to have an azide group capable of reacting with the BCN group of a lysine and functionalised by a chelating agent and a fluorophore as described in Adumeau et al. (Site-Specific Platform-Based Conjugation Strategy for the Synthesis of Dual-Labeled Immunoconjugates for Bi-modal PET/NIRF Imaging of HER2-Positive Tumors. Bioconjugate Chemistry, American Chemical Society, 2022, 33(3), pp. 530-540).

WO2018172543A1 describes a Fab′ fragment bifunctionalised via a tetrazine platform. Conjugation is carried out on the cysteines of the hinge region, which has the disadvantage of causing problems of homogeneity and reproducibility. Moreover, this method requires the presence of a hinge region, which is not present in many antibody fragments, such as a Fab fragment, scFv, Fv fragment, diabody, tribody or tetrabody. This method is therefore limited to the conjugation of Fab′ fragments, with limited homogeneity and reproducibility.

Junutula J R et al. tested the possibility of replacing certain residues of the heavy or light chain fragment of the Fab fragment of the Hu4D antibody with a cysteine to allow site-specific conjugation (Junutula J R et al. Rapid identification of reactive cysteine residues for site-specific labeling of antibody-Fabs, Journal of Immunological Methods, Volume 332, Issues 1-2, 2008, Pages 41-52, https://doi.org/10.1016/j.jim.2007.12.011). The position selected in the heavy chain corresponds to the first amino acid of the constant part, which is position 129 of the heavy chain according to the IMGT nomenclature. Such a position, located in the constant region, can again only be used for antibody fragments comprising part of the constant region, but not for antibody fragments comprising only the variable regions of the antibody, such as an scFv fragment, an Fv fragment, a diabody, a tribody, or a tetrabody. The method adopted is therefore limited to fragments containing at least part of the constant region. Other positions have been identified in the VH domain allowing site-specific conjugation, but these are positions that are not highly conserved within the murine and human VH genes and play a crucial role in the correct folding of the antibody heavy chain (see Example 1 below), which does not enable them to be used in a general method that can be applied to any type of initial antibody and to generate any type of antibody fragment comprising a VH domain. Finally, this document illustrates the extent to which it is difficult to predict the effect of mutation of a cysteine position on conjugation capacity, as the reactivity of thiol groups obtained by cysteine mutation of residues very close to each other is highly variable (see Table 2 of Junutula J R et al.).

There is therefore a need for novel methods for easily obtaining bioconjugates for any type of antibody fragment, for which stability and affinity for the antigen are guaranteed by site-specific grafting. Such a need exists, in particular, for Fab fragments.

The role of the endothelin axis in tumour development is widely described in the scientific literature and is involved in numerous pro-tumour processes.

Endothelin-1 acts as a growth factor in tumour development, resulting in changes in the expression of ETA, ETB and ET-1 in various tumours such as colon, breast, ovarian, prostate, kidney, bladder, lung and ENT cancers, gliomas and melanoma.

More generally, stimulation of the endothelin A receptor by endothelin-1 is thought to induce the proliferation of epithelial tumours. This is the case, for example, with tumour cells from ovarian, cervical, prostate or colon cancer. ET_{B} is expressed in other tumour types such as skin cancer including melanoma, Kaposi's sarcoma, breast cancer, cancer of the vulva and some gliomas. In these various tumours, ETB plays a role in tumour progression and proliferation. When an antagonist of this receptor is used, cell proliferation is inhibited. These antagonists therefore appear to be the candidates of choice for the therapy of these tumours. Various clinical trials of chemical antagonists of endothelin receptors have been or are being conducted in the treatment of cancer, in particular prostate cancer (metastatic or non-metastatic), lung cancer, glioma, metastatic renal cell carcinoma, gallbladder cancer, metastatic melanoma, pancreatic cancer and glioblastoma. These chemical antagonists are well tolerated by the body and have an entirely conventional biodistribution. However, they have shown a mixed clinical efficacy, and may suffer from the presence of high concentrations of endogenous ligand in the tumour. In order to target ETRs and act on the endothelin axis, which is over-activated in tumours, it would seem more appropriate to explore therapeutic alternatives other than ETR antagonists.

Antibodies that bind specifically to an endothelin receptor (ETA or ETB) are therefore interesting candidates, particularly in the form of conjugates with either a therapeutic molecule (for therapy) or a molecule useful in imaging (for diagnosis and aiding surgical tumour resection).

However, endothelin receptors belong to class 1 or family A of G protein-coupled receptors (GPCRs). They therefore comprise 7 transmembrane (TM) domains. Coupled to heterotrimeric G proteins at the carboxyl end of the GPCR, they enable signal transduction within the cell. TM domains are constituted by alpha helices linked by intracellular (i1, i2 and i3) and extracellular (e1, e2 and e3) loops. It is also common to find two retained cysteines between the extracellular e1 and e2 regions (FIG. 9).

The extracellular loops e1, e2 and e3, to which antibodies binding specifically to ETA or ETB typically bind, are particularly short, with a length of only 5 amino acids (e1 of ETA and ETB), 20 or 22 amino acids (e2 of ETA and ETB, respectively) and 6 amino acids (e3 of ETA and ETB) (see FIG. 10). This characteristic is not shared by many cancer antigens, which have much larger extracellular domains. This is the case, in particular, for HER-2 (also known as ERBB2, its extracellular domain corresponds to amino acids 23-652, i.e. 630 amino acids, see Uniprot P04626), TNFR1 (also known as TNR1A or TNFRSF1A, its extracellular domain corresponds to amino acids 30-211, i.e. 182 amino acids, see Uniprot P19438), CD40 (also known as TNR5, its extracellular domain corresponds to amino acids 21-193, 172 amino acids, see Uniprot P25942), CEA6 (also called CEAM6 or CEACAM6, its extracellular domain corresponds to amino acids 35-142, i.e. 108 amino acids, see Uniprot P40199), and GPIIb/Illa (GPIIb is also called ITA2B or ITGA2B, its extracellular domain corresponds to amino acids 32-993, i.e. 962 amino acids, see Uniprot P08514; GPIIIa is also called ITB3 or ITGB3, its extracellular domain corresponds to amino acids 27-718, i.e. 692 amino acids, see Uniprot P05106).

Because of the very short size of the extracellular loops (e1, e2 and e3), antigen-binding constraints are strong in the case of endothelin receptors A and B. These constraints are illustrated, in particular, by the fact that binding of ET-1 to ETB leads to allosteric modifications of the ETB receptor (Shihoya, W., Nishisawa, T., Okuta, A. et al. Activation mechanism of endothelin ETB receptor by endothelin-1. Nature 537, 363-368 (2016)). Consequently, much more than for other antigens, a person skilled in the art would expect that the slightest modification of the structure of an antibody binding specifically to ETA or ETB which affects its conformation is likely to lead to suppression of its recognition of the antigen. This is further illustrated by the fact that a simple chimerisation (replacement of mouse constant regions with human constant regions) of the RB49 antibody led to the obtaining of a non-functional antibody that had lost the ability to bind to the endothelin B receptor (Marie Hautiere et al., (2023) The functionality of a therapeutic antibody candidate restored by a single mutation from proline to threonine in the variable domain, Human Vaccines & Immunotherapeutics, 19:3).

However, there is a need for antibody fragments capable of binding to ETA or ETB, particularly after conjugation with a therapeutic molecule (for therapy) or with a molecule useful in imaging (for diagnosis and aiding surgical tumour resection).

### SUMMARY OF THE INVENTION

The invention addresses this need. In the context of the invention, the inventors have developed mutant Fab fragments from antibodies that bind specifically to ETA or ETB, in which the amino acid residue in position 128 of the heavy chain according to the IMGT nomenclature (i.e. the last amino acid of the VH domain) is substituted by a cysteine, and showed that this mutation does not alter the affinity of the Fab for the antigen, and enables site-specific grafting (i.e. with good homogeneity and reproducibility) of: a) a bi-functionalised tetrazine platform, without alteration of the disulfide point guaranteeing the stability of the Fab fragment and without alteration of the binding to the antigen (see Example 2 below), or b) a chelating agent via a maleimide group (see Example 3 below).

Moreover, because the mutation is in the VH domain of the heavy chain, it can be used broadly to obtain any other antibody fragment with a VH domain, without the need for further development, making the method much more versatile than those described in the prior art.

Finally, the last amino acid of the VH domain is widely conserved in both humans and mice (see Example 1), making the proposed mutation usable in a large number of antibodies.

According to a first aspect, the invention therefore relates to a mutant antibody fragment specifically binding to the endothelin A receptor or endothelin B receptor, comprising a mutated heavy chain variable domain, the amino acid residue at position 128 according to the IMGT nomenclature of the heavy chain variable domain being substituted by a cysteine, provided that said fragment is not a Fab fragment in which the mutated heavy chain fragment corresponds to the amino acid sequence SEQ ID NO: 1 and the light chain fragment corresponds to the amino acid sequence SEQ ID NO: 2.

According to a second aspect, the invention also relates to a nucleic acid molecule or pair of nucleic acid molecules encoding the mutant antibody fragment according to the invention.

According to a third aspect, the invention also relates to a vector comprising the nucleic acid molecule or pair of nucleic acid molecules according to the invention.

According to a fourth aspect, the invention also relates to a host cell, a transgenic non-human animal or a transgenic plant comprising the nucleic acid molecule or pair of nucleic acid molecules according to the invention or the vector according to the invention.

According to a fifth aspect, the invention also relates to a conjugate comprising the mutant antibody fragment according to the invention, bonded to at least one, in particular one or two, molecules of interest, via the cysteine, and more particularly the thiol function of the cysteine, in position 128 of the heavy chain variable domain according to the IMGT nomenclature of the mutant antibody fragment.

According to a sixth aspect, the invention also relates to a method for preparing a conjugate according to the invention comprising the following steps:
a) reducing the disulfide bridge or bridges of the mutant antibody fragment, in particular in the presence of tris(2-carboxyethyl)phosphine (TCEP), to give a reduced mutant antibody fragment,
b) re-oxidising the disulfide bridge or bridges of the mutant antibody fragment, in particular in the presence of dehydroascorbic acid, to give a partially reduced mutant antibody fragment,
c) conjugating, with at least one molecule of interest, the thiol function of the cysteine in position 128 of the heavy chain variable domain according to the IMGT nomenclature of the partially reduced mutant antibody fragment, to give a conjugate according to the invention.

According to a seventh aspect, the invention also relates to an intermediate conjugate comprising the mutant antibody fragment according to the invention, bonded to a reactive chemical group, via the cysteine, and more particularly the thiol function of the cysteine, in position 128 of the heavy chain variable domain according to the IMGT nomenclature, of the mutant antibody fragment.

According to an eighth aspect, the invention also relates to a conjugate according to the invention, for use in:
a) a method for diagnosing a tumour by imaging,
b) a method for surgical treatment by tumour resection guided by fluorescence or nuclear imaging probe,
c) a method for monitoring the effectiveness of an anti-tumour treatment by imaging; or
d) any combination of methods a), b) and c).

### DESCRIPTION OF THE FIGURES

FIG. 1: FIG. 1 shows the antigen binding curves on CHO-ET_{A} cells of the non-mutated Fab fragment of the xiRA63 antibody and a conjugate obtained by random grafting of a bifunctionalised tetrazine with a Zirconium-89 chelating agent (DFO) and a fluorophore (IRDye800).

FIG. 2: FIG. 2 shows the antigen binding curves on CHO-ET_{A} cells of the mutant Fab fragment with a cysteine in position 128 of the heavy chain according to the IMGT nomenclature of the xiRA63 antibody and of a conjugate obtained by site-specific grafting of a bifunctionalised tetrazine with a Zirconium-89 chelating agent (DFO) and a fluorophore (IRDye800).

FIG. 3: FIG. 3 shows the MALDI-TOF mass spectrometry validation of the conjugation of the DFO group to the ThioFab-xiRA63 fragment (light grey line: native ThioFab-xiRA63, dark grey line: ThioFab-xiRA63-DFO).

FIG. 4: FIG. 4 shows the validation of the affinity of the ThioFab-xiRA63-DFO conjugated fragment by flow cytometry. Comparison of the binding curve obtained by flow cytometry between ThioFab-xiRA63 (orange) and ThioFab-xiRA63-DFO (blue). To test their specificity, the antibodies were tested on CHO-ETA (solid line) and CHO-WT (dashed line) not visible in the figure. Data are presented as mean±SEM. Statistical comparisons were made using the paired two-tailed Student's t-test (*p<0.05, **p<0.01, ****p<0.0001). MFI: Median Fluorescence Intensity.

FIG. 5: FIG. 5 shows the evaluation of radiolabelling of [⁸⁹Zr]Zr-ThioFAb-xiRA63-DFO: (A.) HPLC-UV chromatograms of ThioFab-xiRA63-DFO detected at 280 nm before and after radiolabelling with ⁸⁹Zr with a retention time of 18 min for ThioFab-xiRA63-DFO. The second peak after radiolabelling at 25 minutes corresponds to gentisic acid. (B.) HPLC-radiochromatogram of [⁸⁹Zr]-ThioFab-xiRA63. AU: arbitrary units.

FIG. 6: FIG. 6 shows a summary of the in vivo experiment: (A.) experiments and animal sacrifices. Six mice were implanted with gli7 cells and 5 were injected with [89Zr]Zr-ThioFab-xiRA63 (n=5). One mouse was excluded from the experiment and another mouse had no tumour on MRI. Finally, four mice with tumours were injected with [89Zr]Zr-ThioFab-xiRA63. For the biodistribution analysis, all the mice were used, including those without tumours. (B.) Summary table of the injection of [89Zr]Zr-ThioFab-xiRA63 into mice bearing a human glioblastoma tumour. Data are presented as mean±SD FIG. 7: FIG. 7 shows the biodistribution of the radioligand [89Zr]Zr-ThioFab-xiRA63 over time in a nude mouse model. Biodistribution of [89Zr]Zr-ThioFab-xiRA63 extrapolated from PET imaging at different times and in key organs. (n=6 mice). Data are presented as mean±SD, N=6 mice, % DI·cm⁻³: percentage of dose injected per volume of tissue.

FIG. 8: FIG. 8 shows a brain tumour imaged over time by PET with the radioligand [⁸⁹Zr]Zr-ThioFab-xiRA63:_(A.) Quantification of activity in the tumour obtained with antibody [⁸⁹Zr]Zr-ThioFab-xiRA63 at 1 h, 5 h, 24 h, 48 h, 72 h, D7 p.i. (B.) Example of PET imaging of one of the brain tumours imaged with [⁸⁹Zr]Zr-ThioFab-xiRA63 48 h p.i. with the 3 views (transverse, axial and coronal sections). DI.cm⁻³: percentage of dose injected per volume of tissue.

FIG. 9: Classical structure of GPCRs (after Bockaert et al., Bull. Acad. Natle Méd, 2012, 196, no. 9, 1765-1775). Receptors with 7 TM domains linked by 3 extracellular loops (e1, e2 and e3) and 3 intracellular loops (i1, i2, i3). The N-terminal domain is located in the extracellular region, while the C-terminal domain composed of an 8^{th} helix is located on the intracellular side and can interact with intracellular proteins (GIPs for “GPCR Interacting Proteins”).

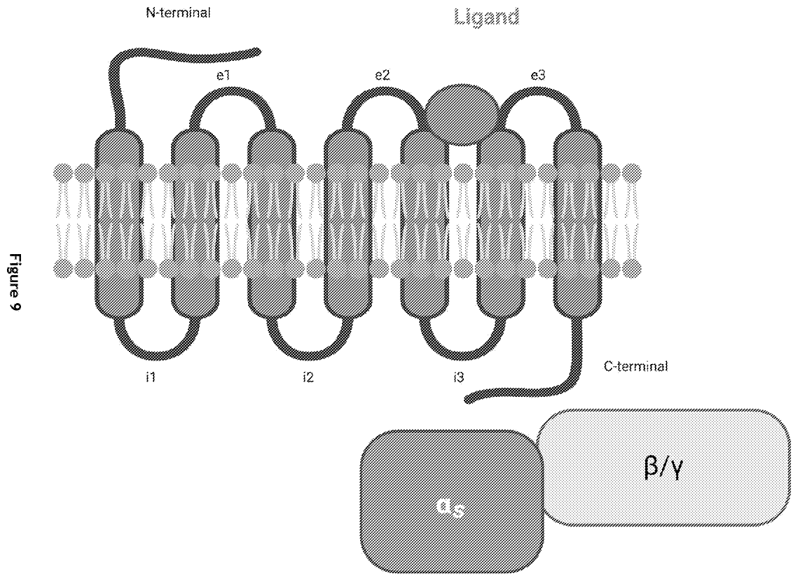

FIG. 10: 2-dimensional (2D) structure of human ETA and ETB obtained by the GPCRdb.

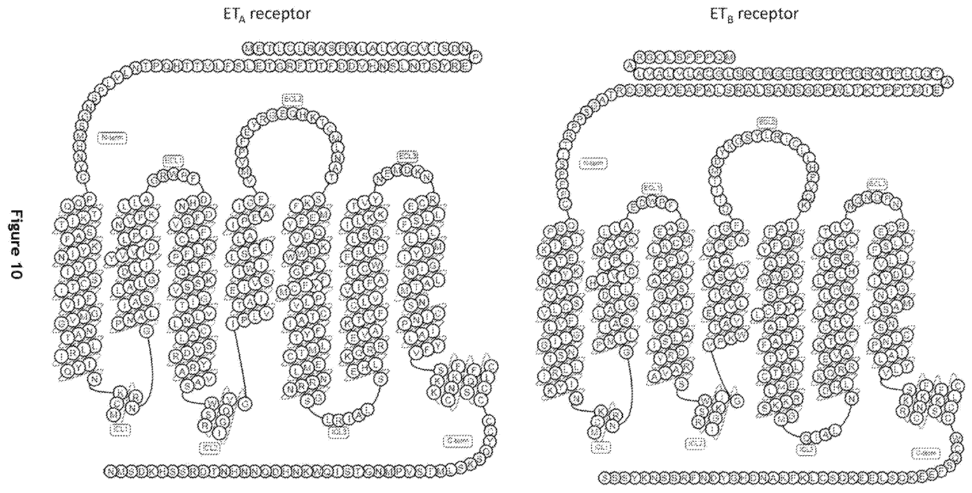

FIG. 11: Competition test protocol. Cells were pre-incubated overnight at 4° C. in the presence of a molar excess (500 nM) of Fab-xiRB49-P125T or ThioFab-xiRB49-P125T antibody fragments. The xiRB49-P125T antibody control range was then added to each cell point. Ranges read by flow cytometry were revealed by Alexa Fluor488-coupled goat polyclonal anti-human IgG secondary antibody (1110120) targeting only the Fc regions of the antibodies. Throughout this experiment, three washes with 1×PBS (pH: 7.4) were performed between each step to remove the non-specific signal.

FIG. 12: Thermal denaturation curves and inflection temperatures Ta and T₁2 of xiRB49-P125T, Fab-xiRB49-P125T, and ThioFab-xiRB49-P125T antibodies.

FIG. 13: Binding curves obtained by flow cytometry of Fab-xiRB49-P125T, and ThioFab-xiRB49-P125T antibodies allowing determination of their apparent Kd and Bmax.

FIG. 14: Binding curves obtained by flow cytometry of the competition assay between xiRB49-P125T and Thio-Fab-xiRB49-P125T or Fab-xiRB49-P125T.

FIG. 15: Binding curves obtained by flow cytometry with a secondary anti-Fd antibody. Summary diagram of secondary antibodies recognising Fab-xiRB49-P125T and ThioFab-xiRB49-P125T.

### DETAILED DESCRIPTION OF THE INVENTION

### General Definitions

The terms “a” or “an” or “one” mean one or more. In other words, when “a” or “an” is used in relation to a feature, it covers both embodiments having the feature of interest only once, and those embodiments having the feature of interest several times. For example, an antibody fragment which comprises a mutated heavy chain variable domain may comprise a single mutated heavy chain variable domain (in particular in the case of an Fv or scFv fragment) or a plurality of mutated heavy chain variable domains (in particular in the case of a Fab fragment, a diaboby, tribody or tetrabody).

In this document, when used to define products, compositions and methods, the terms “comprising” (and any form of “comprising”, such as “comprises” and “comprises”), “having” (and any form of “having”, such as “has” and “has”), “including” (and any form of “including”, such as “includes” and “includes”) or “containing” (and any form of “containing”, such as “contains” and “contains”) are open-ended and do not exclude additional, unmentioned method elements or steps. Thus, a polypeptide “comprises” an amino acid sequence when the amino acid sequence forms part of the final amino acid sequence of the polypeptide. Such a polypeptide can have up to several hundred additional amino acid residues. The term “essentially consisting of” or “essentially constituted by” means the exclusion of other components or stages of any essential importance. Thus, a polypeptide “essentially consists of” an amino acid sequence when such an amino acid sequence is present with possibly only a few additional amino acid residues (for example, a peptide of at most 20 amino acids, such as a label of 6 His×6 histidines, may also be present). “Consisting of” or “constituted by” means excluding more than traces of other components or steps. For example, a polypeptide “consists of” an amino acid sequence when the polypeptide contains no amino acids other than the indicated amino acid sequence. The terms “antibody” or “immunoglobulin” or “Ig”, mean a molecule comprising at least one domain for binding to a given antigen and a constant domain comprising an Fc fragment capable of binding to FcR receptors. In most mammals, such as humans and mice, an antibody is composed of 4 polypeptide chains: 2 heavy chains and 2 light chains joined together by a variable number of disulfide bridges ensuring flexibility in the molecule. Each light chain is constituted by a constant domain (CL) and a variable domain (VL); heavy chains are composed of a variable domain (VH) and 3 or 4 constant domains (CH1 to CH3 or CH1 to CH4) depending on the antibody isotype. In a few rare mammals (camelids, notably camels and lLamas) and cartilaginous fish (notably sharks), antibodies are constituted by just two heavy chains, each heavy chain comprising a variable domain (VH) and a constant region.

The variable domains are involved in antigen recognition, while the constant domains are involved in the biological, pharmacokinetic and effector properties of the antibody.

The variable region differs from one antibody to another. The genes encoding the heavy and light chains of antibodies are generated by recombination of three and two distinct gene segments respectively called VH, DH and JH-CH for the heavy chain and VL and JL-CL for the light chain. The CH and CL segments are not involved in recombination and form the constant regions of the heavy and light chains respectively. Recombinations of the VH-DH-JH and VL-JL segments form the variable regions of the heavy and light chains respectively. The VH and VL regions each have 3 hyper-variable zones or complementarity determining regions (CDRs), called CDR1, CDR2 and CDR3; the CDR3 region being the most variable, since it is located in the recombination zone. These three CDR regions, and in particular the CDR3 region, are located in the part of the antibody that will be in contact with the antigen and are therefore very important for antigen recognition. Thus, the vast majority of antibodies that retain the three CDR regions and each of the heavy and light chains of an antibody retain the antigenic specificity of the original antibody. In a number of cases, an antibody that retains only one of the CDRs, in particular CDR3, also retains the specificity of the original antibody. The CDR1, CDR2 and CDR3 regions are each preceded by the FR1, FR2 and FR3 regions respectively, corresponding to the framework regions FR that vary the least from one VH or VL segment to another. The CDR3 region is likewise followed by an FR4 framework region.

The CDRs of an antibody are defined on the basis of the amino acid sequence of its heavy and light chains according to criteria known to a person skilled in the art. Various methods have been proposed for determining CDRs, and the portion of the amino acid sequence of an antibody heavy or light chain variable region defined as a CDR varies according to the method chosen. A first determination method is that proposed by Kabat et al. (Kabat et al., Sequences of Proteins of Immunological Interest, 5th Ed. Public Health Service, National Institutes of Health, Bethesda, Md. (1991)). In this method, the CDRs are defined by looking for the amino acids responsible for binding the antibody to the antigen. A 2^{nd} method has been proposed by the INTERNATIONAL IMMUNOGENETICS INFORMATION SYSTEM® (IMGT), based in this case on the determination of the hypervariable regions. In this method, a unique numbering system was defined in order to compare variable regions regardless of the antigen receptor, chain type or species (Lefranc et al. 2003). This numbering provides a standardised delimitation of framework regions ((FR1-IMGT: positions 1 to 26, FR2-IMGT: 39 to 55, FR3-IMGT: 66 to 104 and FR4-IMGT: 118 to 128) and regions determining complementarity (CDR1-IMGT: positions 27 to 38, CDR2-IMGT: positions 56 to 65 and CDR3-IMGT: positions 105 to 117). There are still other methods of determination, which will not be detailed here, but are known to a person skilled in the art. A program for determining CDRs from the amino acid sequence of an antibody heavy or light chain according to different nomenclatures (in particular Kabat and IMGT) is the abYsis “Annotate” tool available at http://www.abysis.org/abysis/sequence_input/key_annotation/key_annotation.cgi. In order to determine CDRs according to the IMGT nomenclature only, the IMGT “DomainGapAlign” tool available at https://www.imgt.org/3Dstructure-DB/cgi/DomainGapAlign.cgi is described in two articles by Ehrenmann F. et al. (Ehrenmann F., Kaas Q. and Lefranc M.-P. Nucleic Acids Res, 38:D301-D307 (2010); Ehrenmann, F., Lefranc, M.-P. Cold Spring Harbor Protoc. 6:737-749 (2011)) can also be used.

Unlike variable regions for which the sequence varies greatly from one antibody to another, constant regions are characterised by an amino acid sequence that is very similar from one antibody to another, characteristic of the species and isotype, with possibly a few somatic mutations.

The constant heavy chain region is composed of a C-terminal CH1 domain, a hinge region and CH2 and CH3 or CH2 to CH4 domains (depending on the isotype). The “Fc fragment” is naturally composed of the constant region of the heavy chain excluding the CH1 domain, i.e. the lower hinge region and the constant domains CH2 and CH3 or CH2 to CH4 (depending on the isotype). The positions of the CH1 to CH3 or CH1 to CH4 domains and of the hinge region of the different antibody isotypes of many species are known to a person skilled in the art, and can be found on the IMGT website.

The Fc fragment is glycosylated at the CH2 domain with the presence, on each of the 2 heavy chains, of an N-glycan bonded to the asparagine residue at position 297 (Asn 297).

The following binding domains, located in the Fc, are important for the biological properties of the antibody:
FcRn receptor binding domain, involved in the pharmacokinetic properties (half-life in vivo) of the antibody: Various data suggest that certain residues located at the interface of the CH2 and CH3 domains are involved in binding to the FcRn receptor.
C1q complement protein binding domain, involved in the CDC (complement-dependent cytotoxicity) response: located in the CH2 domain;
FcR receptor-binding domain, involved in phagocytosis or ADCC (antibody-dependent cellular cytotoxicity) responses: located in the CH2 domain.

The light chain constant region (for antibodies with two heavy chains and two light chains) is composed of a single domain called CL.

The terms “monoclonal antibody” or “monoclonal antibody composition” refer to a composition comprising antibody molecules having an identical and unique antigenic specificity. The antibody molecules present in the composition may vary in their degree of post-translational modifications, and in particular in their glycosylation structures or their isoelectric point, but they have all been encoded by the same heavy and light chain sequences and therefore, before any post-translational modification, have the same protein sequence. Some differences in protein sequences, bonded to post-translational modifications (such as cleavage of the C-terminal lysine of the heavy chain, deamidation of asparagine residues and/or isomerisation of aspartate residues), may nevertheless exist between the different antibody molecules present in the composition.

Antibodies can be of several “isotypes”, depending on the nature of their constant region; the constant regions γ, α, μ, ε and δ correspond to the immunoglobulins IgG, IgA, IgM, IgE and IgD respectively. The γ constant regions comprise several subtypes: γ1, γ2, γ3, these three types of constant regions having the particularity of fixing human complement, and γ4, thus creating the IgG1, IgG2, IgG3, and IgG4 sub-types.

The term “chimeric” antibody refers to an antibody which contains a naturally occurring variable region (light chain and heavy chain) derived from an antibody of a given species in association with the light chain and heavy chain constant regions of an antibody of a species heterologous to said given species. Advantageously, if the monoclonal antibody composition for use as a medication according to the invention comprises a chimeric monoclonal antibody, this comprises human constant regions. Starting from a non-human antibody, a chimeric antibody can be prepared using genetic recombination techniques well known to a person skilled in the art. For example, the chimeric antibody could be produced by cloning, for the heavy chain and the light chain, a recombinant DNA comprising a promoter and a sequence encoding the variable region of the non-human antibody, and a sequence encoding the constant region of a human antibody. For methods of preparing chimeric antibodies refer, for example, to Verhoeyen et al. (Verhoeyen et al. BioEssays, 8: 74, 1988).

The term “humanised” antibody refers to an antibody that contains CDR regions derived from an antibody of non-human origin, the other parts of the antibody molecule being derived from one (or more) human antibodies. Moreover, some of the residues in the backbone segments (known as FRs) can be modified to maintain binding affinity (Jones et al. Nature, 321: 522-525, 1986; Verhoeyen et al. Science, 239: 1534-1536, Verhoeyen et al. BioEssays, 8: 74, 1988; 1988; Riechmann et al. Nature, 332: 323-327, 1988). The humanised antibodies according to the invention can be prepared by techniques known to a person skilled in the art, such as CDR grafting, resurfacing, SuperHumanisation, Human string content, FR libraries, Guided selection, FR shuffling and Humaneering technologies, as summarised in the review by Almagro et al. (Almagro et al. Frontiers in Bioscience 13, 1619-1633, Jan. 1, 2008).

The term “human” antibody refers to an antibody that contains only amino acid sequences of human origin. A human antibody recognising a given antigen can be obtained either by immunising a healthy human volunteer or by other methods, such as phage display (see for example Hammers C M, Stanley J R. Antibody phage display: technique and applications. J Invest Dermatol. 2014 February; 134(2):1-5. doi: 10.1038/jid.2013.521) or the immunisation of mammals (in particular mice) in which the loci of the native immunoglobulin genes have been deleted and the loci of the human immunoglobulin genes have been inserted (see for example Bruggemann, M., Osborn, M. J., Ma, B. et al. Human Antibody Production in Transgenic Animals. Arch. Immunol. Ther. Exp. 63, 101-108 (2015). https://doi.org/10.1007/s00005-014-0322-x).

The term “antigen-binding antibody fragment” refers to a fragment of an antibody which retains the antigen-binding domain and therefore has the same antigen specificity as the original antibody. Examples of such antigen-binding antibody fragments include:
for double-chain antibodies (from most mammals, in particular mice and humans): Fv, scFv, Fab, F(ab′)₂, Fab′ fragments, diantibodies (generally called “diabodies”), triantibodies (generally called “tribodies”), tetraantibodies (generally called “tetrabodies”); and
for single-chain antibody fragments (from camelids or cartilaginous fish), VHH fragments (VH domain of a camelid antibody) or VNAR fragments (VH domain of a cartilaginous fish antibody).

The terms “Fv fragment” or “variable fragment” refer to an antibody fragment consisting of non-covalently associated VH and VL domains.

The terms “scFv fragment” or “scFv” or “single-chain variable fragment” or “single-chain FV” refer to a fragment formed by the fusion of the VH domain to the VL domain of an antibody via a peptide called a “linker” constituted by a reduced number of amino acids (generally 15 to 20), either in the VH-linker-VL format or in the VL-linker-VH format. The amino acids in the linker are most often chosen from glycine, serine, threonine, asparagine, alanine and proline, with glycine and serine most often used in the majority. A common format for scFv is VH-linker-VL, where the linker sequence is GGGGSGGGGSGGGGSGGGGS (SEQ ID NO: 23). An scFv generally has a molar mass of around 25 kDa.

The term “Fab fragment” refers to a fragment formed by assembling the part of the heavy chain located upstream of the papain cleavage site (the CH1 domain is included, but the hinge region and the other CH domains are not) and the entire light chain of the antibody of interest. The two chains are linked by a disulfide bridge as in whole Ig. The Fab fragments are monomers of around 50 kDa.

The term “Fab′ fragment” refers to a fragment formed by assembling the part of the heavy chain located upstream of the pepsin cleavage site (the CH1 domain and the hinge region are included, but not the other CH domains) and the entire light chain of the antibody of interest.

The term “F(ab′)₂ fragment” refers to a fragment formed by the pairing of 2 Fab′ fragments by disulfide bridges at the cysteines of the hinge region.

The term “diantibody” or “diabody” refers to a dimer composed of two scFv fragments. The two scFv fragments may be identical (in which case the diabody has a single antigenic specificity) or different (in which case the diabody may be bispecific if the two scFvs recognise different antigens). A diabody generally has a molar mass of around 50 kDa (twice that of an scFv).

The terms “triantibody” or “tribody” or “triabody” refer to a trimer composed of three scFv fragments. The three scFv fragments can be identical (in which case the tribody has a single antigenic specificity) or different (in which case the tribody can be bi-specific or tri-specific depending on the antigenic specificity of the three scFvs). A tribody generally has a molar mass of around 75 kDa (3 times that of an scFv).

The terms “tetraantibody” or “tetrabody” refer to a quadrimer composed of four scFv fragments. The four scFv fragments can be identical (in which case the tetrabody has a single antigenic specificity) or different (in which case the tetrabody can be bi-specific, tri-specific or quadri-specific depending on the antigenic specificity of the four scFvs). A tetrabody generally has a molar mass of around 100 kDa (4 times that of an scFv).

The term “miniantibody” or “minibody” refers to a fragment formed by the fusion of an scFv to a domain that tends to dimerise, and in particular to a CH3 domain. A minibody generally has a molar mass of around 75 kDa.

The terms “nanobody” or “single domain antibody” refer to the variable domain of the heavy chain of a single chain antibody. A nanobody can be, in particular, a VHH or a VNAR. The term “VHH” refers to the variable domain of the heavy chain of a single-chain camelid antibody (in particular of a llama or alpaca). The term “VNAR” refers to the variable domain of the heavy chain of a single-chain antibody of cartilaginous fish (in particular a shark). A VHH or VNAR generally has a molar mass of around 12 to 15 kDa.

An antibody or antibody fragment is said to “specifically bind to” an antigen when it binds to that antigen with a significantly higher binding affinity (e.g. at least 10-fold higher) than that with which it binds to other antigens. Various techniques for measuring the affinity of an antibody for an antigen are well known to a person skilled in the art.

The term “antigen” refers to a substance which, when administered to a subject, can stimulate the immune system to produce antibodies that recognise this substance. In terms of composition, an antigen can be a peptide or a protein, but can also be a nucleic acid, a lipid, a sugar, etc. In terms of origin, an antigen can in particular be a cancer antigen, a pathogenic microorganism antigen (e.g. from a virus, a bacterium, a parasite) or a self-antigen.

The term “cancer antigen” refers to an antigen expressed on the surface of a cancer cell. This cancer antigen is preferably expressed specifically on the surface of cancer cells (e.g. as a result of a tumour-specific mutation), to the exclusion of the subject's healthy cells, expressed predominantly on the surface of cancer cells (i.e. the majority of the subject's healthy cells do not express the cancer antigen), or overexpressed on the surface of cancer cells (i.e. its level of expression on the surface of cancer cells is significantly, for example at least 2, at least 5 or at least 10 times, higher than the level of expression on the surface of healthy cells). Many cancer antigens are known in the prior art.

“Endothelin” refers to a neuropeptide secreted by the vascular endothelium, which has a powerful vasoconstrictive effect on smooth muscle cells. In humans, there are three endothelins called ET-1, ET-2 and ET-3, composed of 21 amino acids and comprising two disulfide bridges between cysteines 1 and 15 and between 3 and 11. ET-1, ENT-2 and ET-3 are derived from three much larger precursors: preproendothelin 1, 2 and 3 (polypeptides of around 200 amino acids).

“Endothelins” or “ETs” are a family of 3 peptides of 21 amino acids, ET-1, ET-2 and ET-3, which bind to two distinct receptors with 7 transmembrane domains: ETA (for “Endothelin subtype A receptor”, also known as EDNRA, information concerning the human gene encoding ETA is available on the NCBI Entrez Gene database under the accession number Gene ID: 1909) and ETB (for ‘Endothelin subtype B receptor’, also known as EDNRB, information about the human gene encoding ETB is available on the NCBI Entrez Gene database under accession number Gene ID: 1910), these two receptors belonging to the G protein-coupled receptor (GPCR) family. The endothelin axis (endothelins and their receptors) is strongly involved in physiological and pathological processes. ET-1 plays a crucial role in the regulation of physiological smooth muscle motility, but ET-1 is also involved in a wide variety of pathologies, including hypertension, heart failure, kidney disorders and infectious diseases. Moreover, the ET axis is overexpressed in cancer of different organs contributing to tumour growth by acting on cell proliferation, survival, migration, differentiation, angiogenesis and inflammatory cell recruitment. ETAs are upregulated in prostate, ovarian and breast cancers while ETBs are overexpressed and downregulated in melanoma, lung, kidney and vulva cancers; ref (Rosanb, L., Spinella, F., Bagnato, A., 2013. Endothelin-1 in cancer: biological implications and therapeutic opportunities. Nat Rev Cancer 13, 637-651).

The term “pathogenic microorganism antigen” refers to an antigen expressed on the surface of a pathogenic microorganism or of cells infected by a pathogenic microorganism.

A “self-antigen” is an antigen naturally expressed by certain healthy cells in a subject. Self-tolerance mechanisms exist, and the immune system does not normally generate antibodies that bind specifically to a self-antigen, but sometimes the control mechanisms are insufficient and a subject produces antibodies against one or more self-antigens. This is known as an autoimmune disease.

The “identity percentages” referred to in the context of the present invention are determined on the basis of a global alignment of the (nucleic or protein) sequences to be compared, i.e. on an alignment of the sequences taken in their entirety over their entire length using any algorithm well known to a person skilled in the art, such as the Needleman and Wunsch algorithm (Needleman and Wunsch. J. Mol. Biol. 48,443-453, 1970). This sequence comparison can be carried out using any software package well known to a person skilled in the art, for example needle software, using a “Gap open” parameter equal to 10.0, a “Gap extend” parameter equal to 0.5 and a “Blosum 62” matrix. The needle software is available, for example, on the ebi.ac.uk website under the name “Align”.

The term “amino acid equivalent” to another amino acid, refers to any amino acid whose structure is close to that of the original amino acid and is therefore unlikely to modify the biological activities of the antibody. Examples of equivalent amino acids are shown below in Table 1:

TABLE 1
Substitutions with equivalent amino acids

| Original amino acid | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (G) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

The term “molecule of interest” refers to a molecule that is useful for an application of the conjugate according to the invention, in particular in the field of diagnostics or in vitro research tools. Such molecules of interest include, in particular, molecules that are detectable or able to become detectable, affinity molecules and pharmacomodulatory molecules.

The term “detectable molecule or molecule able to become detectable” refers to a molecule that can be detected, directly (detectable molecule) or indirectly after modification (molecule able to become detectable), using medical imaging techniques known to a person skilled in the art, in particular isotopic imaging (such as positron emission tomography (PET), single-photon emission computed tomography (SPECT) and Cerenkov Luminescence Imaging (CLI)); optical imaging; fluorescence imaging. These different medical imaging techniques use different types of detectable molecules.

“Isotope imaging” or “nuclear imaging” detects “radionuclides” or “radioisotopes”, i.e. radioactive isotopes of a given atom. In Positron Emission Tomography (PET) and Single Photon Emission Computed Tomography (SPECT), gamma rays from a radionuclide that directly or indirectly emits gamma photons are detected by gamma cameras. In SPECT, the radionuclides used emit a gamma ray directly when they decay, whereas in PET, the radionuclides used emit a positron when they decay, which after annihilation with a surrounding electron, leads to the emission of 2 gamma rays, thus obtained indirectly. In Cerenkov Luminescence Imaging (CLI), Cerenkov Radiation (CR) is detected using optical imaging instruments (at the interface between isotopic imaging and optical imaging). The radioisotopes can be present directly in the probe (detectable molecule), or a probe with a chelating agent (molecule able to become detectable) can be used, which is coupled with a radiometal just before administration. The term “chelating agent” refers to a substance that possesses the ability to fix metallic cations by forming a stable complex. The term “radiometal” refers to a radioactive isotope of a metal atom.

Optical imaging can detect absorbance, in particular that of a chromophore. The term “chromophore” refers to a group of atoms containing one or more double bonds, and forming, with the rest of the molecule, a sequence of conjugated double bonds, thus giving its colour to the molecule comprising it. Optical imaging can also detect Cerenkov radiation (CR).

Fluorescence imaging detects fluorescence emission from a probe comprising a fluorescent molecule or fluorophore. The term “fluorescent molecule” or “fluorophore” refers to a molecule that emits light when exposed to light or other radiation, the light emitted being of a longer wavelength than the light to which the molecule has been exposed. Many fluorophores are molecules with several conjugated aromatic rings or planar and cyclic molecules with one or more π-bonds.

The term “affinity molecule” refers to a molecule that can be selectively and non-covalently bound to another partner chemical group, thus forming a complex.

The term “pharmacomodulating molecule” refers to a chemical molecule, whether synthetic or natural, which can modify the pharmacokinetic properties of another molecule to which it is linked.

The term “halide” means bromide, chloride, iodide or fluoride.

The term “(C₁-Cₓ)alkyl” refers to a saturated, linear or branched hydrocarbon chain containing 1 to x carbon atoms. A (C₁-C₆)alkyl group is therefore a saturated, linear or branched hydrocarbon chain containing 1 to 6, preferably 1 to 4, carbon atoms. Examples include methyl, ethyl, propyl, isopropyl, butyl, isobutyl, sec-butyl, tert-butyl, pentyl and hexyl groups.

The term “cycloalkane” refers to a saturated monocyclic or polycyclic hydrocarbon comprising one or more, in particular 1 or 2, rings, which rings may be fused (i.e. they share a bond between two carbon atoms) or bridged (i.e. they share a sequence of at least three carbon atoms). Preferably, the cycloalkane will comprise 3 to 9 carbon atoms. Preferably it will be cyclopropane, cyclohexane, cyclooctane, bicyclononane or norbornane.

The term “heterocycloalkane” refers to a cycloalkane in which one or more, in particular 1, 2, 3 or 4, carbon atoms have each been replaced by a heteroatom chosen from O, S and N, in particular 0 or N. These include, in particular, piperidine, 1,2-piperazine, 1,4-piperazine, pyrrolidine and morpholine.

The term “cycloalkene” refers to a monocyclic or polycyclic hydrocarbon comprising at least one double bond and comprising one or more, in particular 1 or 2 rings, which rings may be fused (i.e. they share a bond between two carbon atoms) or bridged (i.e. they share a sequence of at least three carbon atoms). Preferably, the cycloalkene will comprise 3 to 10 carbon atoms. Preferably, it will be cyclopropene, cyclohexene, cyclooctene, bicyclononene or norbornene.

The term “heterocycloalkene” refers to a cycloalkene in which one or more, in particular 1, 2, 3 or 4, carbon atoms have been replaced respectively by a heteroatom chosen from 0, S and N, in particular 0 or N. Examples include, in particular, 3-pyrroline and 1,2,3,6-tetrahydropyridazine.

The term “cycloalkyne” refers to a monocyclic or polycyclic hydrocarbon comprising at least one triple bond and comprising one or more, in particular 1 or 2 rings, which rings may be fused (i.e. they share a bond between two carbon atoms) or bridged (i.e. they share a sequence of at least three carbon atoms). Preferably, the cycloalkyne will comprise 3 to 9 carbon atoms. Preferably it will be cyclooctyne or bicyclononyne.

The term “heterocycloalkyne” refers to a cycloalkyne in which one or more, in particular 1, 2, 3 or 4, carbon atoms have been replaced respectively by a heteroatom chosen from 0, S and N, in particular 0 or N. This may be, in particular, azacyclooctyne.

The term “aromatic ring” refers to an monocyclic or polycyclic aromatic hydrocarbon comprising one or more, in particular 1 or 2 rings joined together. It will advantageously comprise 6 to 14, in particular 6 to 10, carbon atoms. It may be a benzene or a naphthalene. When an aromatic ring is joined to another ring, this means that the two rings share a bond between two carbon atoms.

The term “heteroaromatic ring” refers to an aromatic monocyclic or polycyclic ring comprising one or more, in particular 1, 2 or 3 rings joined together, the atoms making up the ring or rings comprising one or more, in particular 1, 2, 3 or 4 heteroatoms chosen from O, S and N, in particular 0 and N, the other atoms making up the ring or rings being carbon atoms. Advantageously, the ring or rings will be formed of 5 to 15 atoms, in particular 5 to 10 atoms. Preferably, each cycle will be a 5-member or 6-member cycle. These may include, in particular, tetrazine, triazine, 1,2,3-triazole, pyridazine, thiophene, thiazole, thiadiazole and pyridine.

### Mutant Antibody Fragment

In a first aspect, the invention relates to a mutant antibody fragment specifically binding to the endothelin A receptor or endothelin B receptor, comprising a mutated heavy chain variable domain, the amino acid residue at position 128 according to the IMGT nomenclature of the heavy chain variable domain being substituted by a cysteine, provided that said fragment is not a Fab fragment in which the mutated heavy chain fragment corresponds to the amino acid sequence SEQ ID NO: 1 and the light chain fragment corresponds to the amino acid sequence SEQ ID NO: 2.

### Type of Fragment

The invention covers any type of mutant antigen-binding antibody fragment comprising a mutated heavy chain variable domain as defined herein.

In particular, the invention covers both a fragment:
a) which further comprises a light chain variable domain (from antibodies with two heavy chains and two light chains) and
b) which does not comprise a light chain variable domain (derived from single chain antibodies comprising only a heavy chain).

However, fragments which also comprise a light chain variable domain (from antibodies with two heavy chains and two light chains) are preferred, because the amino acid sequences of the heavy and light chains of many antibodies with two heavy chains and two light chains against many antigens, are available in the prior art and therefore accessible to a person skilled in the art.

Of the fragments which further comprise a light chain variable domain (derived from antibodies having two heavy chains and two light chains), the mutant antibody fragment according to the invention can be any type of fragment having a mutated heavy chain (VH) variable domain and a light chain (VH) variable domain, but can advantageously be chosen from a Fab fragment, an scFv fragment, an Fv fragment, a diabody, a tribody, a tetrabody and a minibody. In particular, the Fab, scFv and diabody fragments are preferred (especially the Fab fragment), because they have the lowest molecular weight and are therefore the most likely to cross the blood-brain barrier, with a short in vivo lifespan suitable for diagnosis.

Among the fragments which do not comprise a light chain variable domain (derived from single chain antibodies comprising only a heavy chain), the mutant antibody fragment according to the invention may be, in particular, a nanobody, advantageously chosen from VHH and VNAR fragments.

The substitution of a cysteine for the amino acid residue in position 128 according to the IMGT nomenclature of the heavy chain variable domain, is intended to enable selective conjugation on this cysteine. The inventors have shown that, provided a specific method is used, it is possible to carry out a specific conjugation on this cysteine in a Fab fragment, without affecting the other cysteines present in this fragment.

The mutant antibody fragments according to the invention are intended to be conjugated to groups that are useful for in vivo use (diagnosis in particular), in particular in humans. Consequently, the mutant antibody fragment according to the invention is advantageously chimeric (if it contains a part of the constant region, such as a Fab, then this part is human), humanised or human. For use in a non-human animal, a mutant antibody fragment according to the invention will be used by analogy (if it contains a part of the constant region, such as a Fab, then this part is from the species of the animal in which the fragment is used), modified to replace the framework regions of the variable domains with regions from the species of the animal in which the fragment is used, or entirely from the species in which the fragment is used.

### Antigen Recognised by the Mutant Antibody Fragment

The antigen recognised by the mutant antibody fragment (in particular Fab, scFv or diabody, in particular Fab) according to the invention is of little importance, the invention being applicable regardless of the antigen to which the original antibody specifically binds.

However, the mutant antibody fragments according to the invention are intended to be conjugated to groups that are useful for in vivo use (diagnosis in particular), and the antigen will therefore advantageously be chosen from among antigens of diagnostic interest. In particular, the mutant antibody fragment (in particular Fab, scFv or diabody, in particular Fab) according to the invention binds specifically to a cancer antigen, a pathogenic microorganism antigen (in particular a viral, bacterial or parasitic antigen), or a self antigen.

A mutant antibody fragment (in particular Fab, scFv or diabody, in particular Fab) according to the invention that binds specifically to a cancer antigen will be useful for in vivo uses in the diagnosis, and possibly the treatment, of cancers, and is particularly preferred.

A mutant antibody fragment (in particular Fab, scFv or diabody, in particular Fab) according to the invention can bind specifically to any cancer antigen of interest. Cancer antigens of interest include, in particular, CD19, CD20, CD22, CD38, HER2, CEA, PD1, PD-L1, CTLA-4, B7-H3 (also known as CD276), EpCAM, Folate receptor alpha, BCMA, LAG-3, gp100, GD2, TROP-2, Nectin-4, CD79b, CCR4, PDGRFα, SLAMF7, EGFR, and endothelin receptor A or B.

The mutant antibody fragment (in particular Fab, scFv or diabody, in particular Fab) according to the invention can bind specifically to the endothelin A receptor or the endothelin B receptor.

A mutant antibody fragment (in particular Fab, scFv or diabody, in particular Fab) according to the invention which binds specifically to a pathogenic microorganism antigen will be useful for in vivo uses in the diagnosis, and possibly the treatment, of an infection by the pathogenic microorganism from which the antigen originates.

A mutant antibody fragment (in particular Fab, scFv or diabody, in particular Fab) according to the invention which binds specifically to a self-antigen will be useful for in vivo uses in the diagnosis, and possibly the treatment, of an autoimmune disease involving the production of antibodies against a self-antigen.

Preferred Antibodies from which the Mutant Antibody Fragment is Derived

The inventors have demonstrated that it is possible to specifically conjugate a mutant Fab fragment derived from an RA63 antibody to endothelin receptor A in which the alanine residue at position 128 according to the IMGT nomenclature of the heavy chain variable domain has been substituted by a cysteine.

As explained in Example 1 below, the residue at position 128 according to the IMGT nomenclature of the heavy chain variable domain of antibodies with a murine or human FR4 region is, unless there is a somatic mutation at this position, a serine (in most cases) or an alanine.

The mutant antibody fragment (in particular Fab, scFv or diabody, in particular Fab) according to the invention is therefore advantageously derived (by substitution of the residue at position 128 according to the IMGT nomenclature of the antibody heavy chain variable domain by a cysteine) from an antibody fragment whose amino acid residue at position 128 according to the IMGT nomenclature of the heavy chain variable domain is a serine or an alanine.

The mutant antibody fragment (in particular Fab, scFv or diabody, in particular Fab) according to the invention can therefore be derived (by substitution of the residue, at position 128, according to the IMGT nomenclature of the antibody heavy chain variable domain, with a cysteine) from an antibody fragment whose amino acid residue at position 128 according to the IMGT nomenclature of the heavy chain variable domain is a serine.

Alternatively, the mutant antibody fragment (in particular Fab, scFv or diabody, in particular Fab) according to the invention can therefore be derived (by substitution of the residue, at position 128 according to the IMGT nomenclature of the antibody heavy chain variable domain, with a cysteine) from an antibody fragment whose amino acid residue, at position 128 according to the IMGT nomenclature of the heavy chain variable domain, is an alanine.

In this case, the mutant antibody fragment according to the invention can be derived (by substitution of the residue, at position 128 according to the IMGT nomenclature of the antibody heavy chain variable domain, with a cysteine) from a fragment of the RB49 or RA63 antibody or a variant thereof, which bind specifically to endothelin B receptor.

The RB49 antibody binds specifically to endothelin B receptor and has 6 CDRs defined according to the IMGT nomenclature as:

heavy chain:
(SEQ ID NO: 3, CDR1-H)
GYTFISYW,
(SEQ ID NO: 4, CDR2-H)
IDPDSGGT
and
(SEQ ID NO: 5, CDR3-H)
AREGDYAWFAY,
and
light chain:
(SEQ ID NO: 6, CDR1-L)
QSIVHSNGNTY,
(SEQ ID NO: 7, CDR2-L)
KVS
and
(SEQ ID NO: 8, CDR3-L)
FQGSHVPWT.

The RA63 antibody binds specifically to endothelin A receptor and has 6 CDRs defined according to the IMGT nomenclature as:

heavy chain:
(SEQ ID NO: 9)
GFTFNIYA,
(SEQ ID NO: 10)
IRSKSNNYAT
and
(SEQ ID NO: 11)
VSSYYSGSFFAY,
and
light chain:
(SEQ ID NO: 12)
SQSIVYSNGKIYL,
(SEQ ID NO: 13)
KVS
and
(SEQ ID NO: 14)
FQGSHLPLT.

Thus, in a preferred embodiment, the mutant antibody fragment (in particular Fab, scFv or diabody, in particular Fab) according to the invention binds specifically to the endothelin B receptor, and comprises (in addition to the substitution of the amino acid residue at position 128 according to the IMGT nomenclature of the heavy chain variable domain by a cysteine):
a heavy chain variable domain (VH) comprising three heavy chain CDRs, namely CDR1-H, CDR2-H and CDR3-H having according to the IMGT nomenclature for respective sequences GYTFISYW (SEQ ID NO: 3), IDPDSGGT (SEQ ID NO: 4) and AREGDYAWFAY (SEQ ID NO: 5), or a sequence with at least 80% identity with GYTFISYW (SEQ ID NO: 3), IDPDSGGT (SEQ ID NO: 4) or AREGDYAWFAY (SEQ ID NO: 5), and
a light chain (VL) variable domain comprising three light chain CDRs, namely CDR1-L, CDR2-L and CDR3-L having according to the IMGT nomenclature for respective sequences QSIVHSNGNTY (SEQ ID NO: 6), KVS (SEQ ID NO: 7) and FQGSHVPWT (SEQ ID NO: 8) or a sequence with at least 80% identity with QSIVHSNGNTY (SEQ ID NO: 6), KVS (SEQ ID NO: 7) or FQGSHVPWT (SEQ ID NO: 8).

When at least one of the 6 CDRs defined in the above paragraph does not have 100% identity with SEQ ID NO: 3 to 8, the one or more amino acids which differ from the native sequence are preferably substituted by an equivalent amino acid, so as to maintain affinity for the ETB antigen.

Still more preferably, the mutant antibody fragment (in particular Fab, scFv or diabody, in particular Fab) according to the invention binds specifically to the endothelin B receptor, and also comprises (in addition to the CDRs indicated above which correspond to that of the RB49 antibody or to variants thereof):
a VH domain having the sequence SEQ ID NO: 15 (QVQLQQPGAALVKPGASVKLSCKASGYTFISYWMLWVKQRPGRGLEWIGRIDPDSGGTKYNEKFKSKATL TVDKSSSTAYMQLSSLTSEDSAVYYCAREGDYAWFAYWGQGTLVPVSA) or a sequence having at least 80% identity with SEQ ID NO: 15, such as the sequence SEQ ID NO: 27 (QVQLQQPGAALVKPGASVKLSCKASGYTFISYWMLWVKQRPGRGLEWIGRIDPDSGGTKYNEKFKSKATL TVDKSSSTAYMQLSSLTSEDSAVYCAREGDYAWFAYWGQGTLVTVSC), and
a VL domain having the sequence SEQ ID NO: 16 (DVLMTQTPLSLPVSLGDQASISCRSSQSIVHSNGNTYLEWYLQKPGQSPKLLIYKVSNRFSGVPDRFSGSGT DFTLKISRVEAEDLGVYCFQGSHVPWTFGGGTKLEIK) or a sequence having at least 80% identity with SEQ ID NO: 16.

The sequences of the VH and VL domains most important for antigen binding are those of the 6 CDRs. Therefore, in a preferred embodiment, the mutant antibody fragment (in particular Fab, scFv or diabody, in particular Fab) according to the invention binds specifically to the endothelin B receptor, and comprises:
a VH domain having a sequence with at least 80% identity to SEQ ID NO: 15 and comprising three heavy chain CDRs, namely CDR1-H, CDR2-H and CDR3-H having according to the IMGT nomenclature for respective sequences GYTFISYW (SEQ ID NO: 3), IDPDSGGT (SEQ ID NO: 4) and AREGDYAWFAY (SEQ ID NO: 5); and
a VL domain possessing a sequence having at least 80% identity with SEQ ID NO: 16 comprising three light chain CDRs, namely CDR1-L, CDR2-L and CDR3-L having according to the IMGT nomenclature for respective sequences QSIVHSNGNTY (SEQ ID NO: 6), KVS (SEQ ID NO: 7) and FQGSHVPWT (SEQ ID NO: 8).

In a particularly advantageous manner, the mutant antibody fragment (in particular Fab, scFv or diabody, in particular Fab) according to the invention binds specifically to the endothelin B receptor, and comprises:
a VH domain with the sequence SEQ ID NO: 15 or SEQ ID NO: 27, and
a VL domain with the sequence SEQ ID NO: 16.

In another preferred embodiment, the mutant antibody fragment (in particular Fab, scFv or diabody, in particular Fab) according to the invention binds specifically to the endothelin B receptor, and comprises (in addition to the substitution of the amino acid residue at position 128 according to the IMGT nomenclature of the heavy chain variable domain by a cysteine):
a VH domain comprising three heavy chain CDRs, namely CDR1-H, CDR2-H and CDR3-H having according to the IMGT nomenclature for respective sequences GFTFNIYA (SEQ ID NO: 9), IRSKSNNYAT (SEQ ID NO: 10) and VSSYYSGSFFAY (SEQ ID NO: 11), or a sequence with at least 80% identity with GFTFNIYA (SEQ ID NO: 9), IRSKSNNYAT (SEQ ID NO: 10) or VSSYYSGSFFAY (SEQ ID NO: 11), and
a VL domain comprising three light chain CDRs, namely CDR1-L, CDR2-L and CDR3-L, having according to the IMGT nomenclature for respective sequences SQSIVYSNGKIYL (SEQ ID NO: 12), KVS (SEQ ID NO: 13) and FQGSHLPLT (SEQ ID NO: 14) or a sequence with at least 80% identity with SQSIVYSNGKIYL (SEQ ID NO: 12), KVS (SEQ ID NO: 13) or FQGSHLPLT (SEQ ID NO: 14).

When at least one of the 6 CDRs defined in the above paragraph does not have 100% identity with SEQ ID NO: 9 to 14, the one or more amino acids which differ from the native sequence are preferably substituted by an equivalent amino acid, so as to maintain affinity for the ETA antigen.

Still more preferably, the mutant antibody fragment (in particular Fab, scFv or diabody, in particular Fab) according to the invention binds specifically to the endothelin A receptor, and also comprises (in addition to the CDRs indicated above which correspond to that of the RA63 antibody or to variants thereof):
a VH domain having the sequence SEQ ID NO: 17 (EVQLVESGGGLVQPKGSLKLSCAASGFTFNIYAMNWIRQAPGKGLEWIARIRSKSNNYATYADSVKDRFTIS RDDSQNMVYLQMNNLKTEDTAMYYCVSSYYSGSFFAYWGQGTLVTVSA) or a sequence having at least 80% identity with SEQ ID NO: 17 such as the sequence SEQ ID NO: 28 (EVQLVESGGGLVQPKGSLKLSCAASGFTFNIYAMNWIRQAPGKGLEWIARIRSKSNNYATYYADSVKDRFTI SRDDSQNMVYLQMNNLKTEDTAMYYCVSSYYSGSFFAYWGQGTLVTVSC), and
a VL domain having the sequence SEQ ID NO: 18 (DVLMTQTPLSLPVSLGDQASISCRSSQSIVYSNGKIYLEWYLQKPGQSPKLLIYKVSNRFSGVPDRFSGSGTD FTLKISRVEAEDLGVYCFQGSHLPLTFGAGTKLELKR) or a sequence having at least 80% identity with SEQ ID NO: 18.

The sequences of the VH and VL domains most important for antigen binding are those of the 6 CDRs. Therefore, in a preferred embodiment, the mutant antibody fragment (in particular Fab, scFv or diabody, in particular Fab) according to the invention binds specifically to the endothelin A receptor, and comprises:
a VH domain having a sequence with at least 80% identity to SEQ ID NO: 17 and comprising three heavy chain CDRs, namely CDR1-H, CDR2-H and CDR3-H having according to the IMGT nomenclature for respective sequences GFTFNIYA (SEQ ID NO: 9), IRSKSNNYAT (SEQ ID NO: 10) and VSSYYSGSFFAY (SEQ ID NO: 11); and
a VL domain possessing a sequence having at least 80% identity with SEQ ID NO: 18 comprising three light chain CDRs, namely CDR1-L, CDR2-L and CDR3-L having according to the IMGT nomenclature for respective sequences SQSIVYSNGKIYL (SEQ ID NO: 12), KVS (SEQ ID NO: 13) and FQGSHLPLT (SEQ ID NO: 14).

In a particularly advantageous manner, the mutant antibody fragment (in particular Fab, scFv or diabody, in particular Fab) according to the invention binds specifically to the endothelin B receptor, and comprises:
a VH domain with the sequence SEQ ID NO: 17 or SEQ ID NO: 28, and
a VL domain with the sequence SEQ ID NO: 18.

The RB49 and RA63 antibodies from which the mutant antibody fragment according to the invention is advantageously derived are chimeric antibodies, and when the mutant antibody fragment according to the invention contains part of the constant regions (for example for a Fab fragment), this is preferably human.

In particular, the mutant antibody fragment according to the invention may in particular be chosen from:
a) a mutant Fab fragment binding specifically to the endothelin B receptor and comprising a heavy chain fragment of sequence SEQ ID NO: 19 (QVQLQQPGAALVKPGASVKLSCKASGYTFISYWMLWVKQRPGRGLEWIGRIDPDSGGTKYNEKFKSKATL TVDKSSSTAYMQLSSLTSEDSAVYYCAREGDYAWFAYWGQGTLVTVSCASTKGPSVFPLAPSSKSTSGGAL GCLVKDYFPEPVTVSWNSGALTSGVHTFPAVLQSSGLYSLSSVVTVPSSSLGTQTYNHVKPSNTKVDKKVEP KSCDKTH) and a light chain of sequence SEQ ID NO: 20 (DVLMTQTPLSLPVSLGDQASISCRSSQSIVHSNGNTYLEWYLQKPGQSPKLLIYKVSNRFSGVPDRFSGGSG TDFTLKISRVEAEDLGVYYCFQGSHVPWTFGGGTKLEIKRTVAAPSVFIFPPSDEQLKSGTASVVCLLNNFYP REAKVQWKVDNALQSGNSQESVTEQDSKDSTYSLSSTLTLSKADYEKHKVYACEVTHQGLSSPVTKSFNRG EC); and
b) a mutant Fab fragment binding specifically to the endothelin A receptor and comprising a heavy chain fragment of sequence SEQ ID NO: 21 (EVQLVESGGGLVQPKGSLKLSCAASGFTFNIYAMNWIRQAPGKGLEWIARIRSKSNNYATYYADSVKDRFTI SRDDSQNMVYLQMNNLKTEDTAMYYCVSSYYSGSFFAYWGQGTLVTVSCASTKGPSVFPLAPSSKSTSGGT AALGCLVKDYFPEPVTVSWNSGALTSGVHTFPAVLQSSGLYSLVSSVTVPSSSLGTQTYICNVNHKPSNTKV DKKVEPKSCDKTH) and a light chain of sequence SEQ ID NO: 22 (DVLMTQTPLSLPVSLGDQASISCRSSQSIVYSNGKIYLEWYLQKPGQSPKLLIYKVSNRFSGVPDRFSGSGTD FTLKISRVEAEDLGVYYCFQGSHLPLTFGAGTKLELKRTVAAPSVFIFPPSDEQLKSGTASVVCLLNNFYPRE AKVQWKVDNALQSGNSQESVTEQDSKDSTYSLSSTLTLSKADYEKHKVYACEVTHQGLSSPVTKSFNRGEC).

Alternatively, the mutant antibody fragment according to the invention may in particular be chosen from.
a) an scFv fragment specifically binding to the endothelin B receptor comprising or constituted by the amino acid sequence SEQ ID NO: 25 (DVLMTQTPLSLPVSLGDQASISCRSSQSIVHSNGNTYLEWYLQKPGQSPKLLIYKVSNRFSGVPDRFSGGSG TDFTLKISRVEAEDLGVYYCFQGSHVPWTFGGGTKLEIKGGGGSGGGGSGGGGSQVQQLQQPGAALVKP GASVKLSCKASGYTFISYWMLVKQRPGRGLEWIGRIDPDSGGTKYNEKFKSKATLVDKSSSTAYMQLSSLTS EDSAVYYCAREGDYAWFAYWGQGTLVTVSC); and
b) an scFv fragment specifically binding to the endothelin A receptor comprising or constituted by the amino acid sequence SEQ ID NO: 26

(DVLMTQTPLSLPVSLGDQASISCRSSQSIVYSNGKIYLEWYLQKP
GQSPKLLIYKVSNRFSGVPDRFSGSGTDTFTLKISRVEAEDLGVYY
CFQGSHLPLTFGAGTKLELKGGGGSGGGGSGGGGSGGGGSEVOLVE
SGGGLVQPKGSLKLSCAASGFTFNIYAMNWIRQAPGKGLEWIARIR
SKSNNYATYYADSVKDRFTISRDDSQNMVYLQMNNLKTEDTAMYYC
VSSYYSGSFFAYWGQGTLVTVSC).

### Excluded Fragments

The mutant antibody fragment according to the invention is not a Fab fragment in which the mutated heavy chain fragment corresponds to the amino acid sequence SEQ ID NO: 1 and the light chain fragment corresponds to the amino acid sequence SEQ ID NO: 2. Such a Fab fragment would correspond to the Fab fragment of the Hu4D5 antibody (a humanised antibody that binds specifically to HER2), in which the serine residue at position 128 according to the IMGT nomenclature of the heavy chain variable domain is substituted by a cysteine.

More broadly, a mutant antibody fragment according to the invention may not be an antibody fragment whose heavy chain (VH) variable domain corresponds to the amino acid sequence SEQ ID NO: 1. This excludes all mutant antibody fragments for which the VH domain corresponds to the VH domain of the Hu4D5 antibody (a humanised antibody that binds specifically to HER2), in which the serine residue at position 128 according to the IMGT nomenclature of the heavy chain variable domain is substituted by a cysteine (regardless of the type of fragment or the sequence of any light chain).

Even more broadly, a mutant antibody fragment according to the invention may not be a fragment of an antibody binding to the human HER2 antigen.

Alternatively, the mutant antibody fragment according to the invention is not derived (by substitution of the residue at position 128 according to the IMGT nomenclature of the antibody heavy chain variable domain by a cysteine) from an antibody fragment whose amino acid residue at position 128 according to the IMGT nomenclature of the heavy chain variable domain is a serine.

### Nucleic Acid Molecule or Pair of Nucleic Acid Molecules Encoding the Mutant Antibody Fragment According to the Invention

The present invention also relates to a nucleic acid molecule or pair of nucleic acid molecules encoding the mutant antibody fragment according to the invention.

Such a molecule or pair of molecules is useful, in particular, for producing the mutant antibody fragment according to the invention.

Depending on the desired mutant antibody fragment, this may comprise a single chain (for example, fragments of type scFv, diabody, tribody, tetrabody, minibody, VHH, VNAR) or two chains (for example, fragments of type Fv, Fab, Fab′, F(ab′)2).

When the mutant antibody fragment according to the invention comprises a single chain, a single nucleic acid molecule is preferably used to encode this chain.

When the mutant antibody fragment according to the invention comprises two chains, the two chains can be encoded either by a single nucleic acid molecule or by two nucleic acid molecules.

Due to the degeneracy of the genetic code, all the different nucleic sequences encoding a particular amino acid sequence of a mutant antibody fragment according to the invention are within the scope of the invention.

In particular, the sequence of a nucleic acid according to the invention may have been optimised to promote its expression in a host cell, a transgenic non-human animal or a transgenic plant of interest. More specifically, there are generally several combinations of three nucleotides encoding the same amino acid (except for methionine and tryptophan), known as synonymous codons. However, some of these combinations are, in general, used preferentially by a given cell or organism (this is known as genetic code usage bias). This preference depends in particular on the organism that produces the cell or from which it originates. Consequently, when a protein derived from one or more organisms is produced in a heterologous organism or in a cell of such a heterologous organism, it may be useful to modify the nucleic sequence encoding the protein so as to use predominantly the codons preferred by the heterologous organism. Data are available in the literature concerning the use of preferred codons by different species, and a person skilled in the art knows how to optimise the expression of a given protein in an organism or a cell of a heterologous organism.

The nucleic acid molecule or molecules encoding the mutant antibody fragment according to the invention may also comprise a nucleic acid sequence encoding a signal peptide at the N-terminus of the one or more chains of the mutant antibody fragment according to the invention.

### Vector

The present invention also relates to a vector comprising the nucleic acid molecule or pair of nucleic acid molecules according to the invention, as described above.

Such a vector is also useful for producing the mutant antibody fragment according to the invention, which can be used to transform (stably or transiently) a host cell which will then produce the mutant antibody fragment according to the invention.

Such a vector comprises the elements necessary for the expression of said nucleic sequence, and in particular a promoter, a transcription initiation codon, termination sequences and appropriate transcription regulation sequences. These elements vary according to the host used for the expression and are easily chosen by a person skilled in the art in light of their general knowledge. In particular, for a vector intended for expression in eukaryotic cells, the vector advantageously comprises a “Kozak sequence”, i.e. a conserved sequence found on eukaryotic messenger RNAs at the translation start site, around the AUG start codon (generally GCCGCCRCCATGG (SEQ ID NO: 24), the translation start codon being underlined, R=A or G).

The vector may be a plasmid or viral vector. Examples of plasmid and viral vectors suitable for expressing an antibody fragment are known to a person skilled in the art (see in particular Sandomenico, A., Sivaccumar, J. P., Ruvo, M., 2020. Evolution of Escherichia coli Expression System in Producing Antibody Recombinant Fragments. International Journal of Molecular Sciences 21, 6324; Spadiut, O., Capone, S., Krainer, F., Glieder, A., Herwig, C., 2014. Microbials for the production of monoclonal antibodies and antibody fragments. Trends in Biotechnology 32, 54-60).

### Host Cells, Transgenic Non-Human Animal or Transgenic Plant

The present invention also relates to a host cell, a transgenic non-human animal or a transgenic plant comprising the nucleic acid molecule or pair of nucleic acid molecules according to the invention or the vector according to the invention, as described above.

Such a host cell, such a transgenic non-human animal and such a transgenic plant are also useful for producing the mutant antibody fragment according to the invention, these expressing the mutant antibody fragment according to the invention.

The host cell may be of prokaryotic or eukaryotic origin, and may in particular be selected from bacterial, insect, plant, yeast or mammalian cells. The mutant antibody fragment according to the invention can then be produced by culturing the host cell under suitable conditions. A host cell according to the invention can be obtained, in particular, by transforming a cell line with a vector according to the invention and separating the various cell clones obtained. The transformed cell line is preferably of eukaryotic origin, and may in particular be chosen from insect, plant, yeast or mammalian cells. Cell lines suitable for the production of the mutant antibody fragment according to the invention include, in particular, Escherichia coli among bacteria, Saccharomyces cerevisiae and Pichia pastoris among yeasts, and CHO, NS0, Sp2/0, HEK293, and PER.C6 cell lines among mammalian cells.

A transgenic non-human animal according to the invention can be obtained by direct injection of the gene or genes of interest (here the nucleic acid molecule or pair of nucleic acid molecules according to the invention) into a fertilised egg (Gordon et al., 1980 Proc Natl Acad Sci USA.; 77:7380-4). A transgenic non-human animal can also be obtained by introducing the gene or genes of interest (here the nucleic acid molecule or pair of nucleic acid molecules according to the invention) into an embryonic stem cell and preparing the animal by a chimera aggregation method or a chimera injection method (see Manipulating the Mouse Embryo, A Laboratory Manual, Second edition, Cold Spring Harbor Laboratory Press (1994); Gene Targeting, A Practical Approach, IRL Press at Oxford University Press (1993)). A transgenic non-human animal can also be obtained by a cloning technique in which a nucleus, into which the gene or genes of interest (here the nucleic acid molecule or pair of nucleic acid molecules according to the invention) has been introduced, is transplanted into an enucleated egg (Ryan et al., 1997 Science; 278: 873-876; Cibelli et al., 1998 Science, 280: 1256-1258; WO00/26357). The mutant antibody fragment according to the invention can then be accumulated in the transgenic animal and harvested, in particular from the milk or eggs of the animal. For the production of antibodies in the milk of transgenic non-human animals, preparation methods are described, in particular, in WO90/04036, WO95/17085, WO01/26455, WO2004/050847, WO2005/033281, WO2007/048077. Methods for purifying proteins of interest from milk are also known (see WO01/26455, WO2007/106078). Transgenic non-human animals of interest include mice, rabbits, rats, goats, cattle (especially cows) and poultry (especially chickens).

A transgenic plant according to the invention can be chosen from any plant that allows the production of antibody fragments. Many antibodies have already been produced in transgenic plants and the technologies required to obtain a transgenic plant expressing an antibody of interest and to recover the antibody are well known to a person skilled in the art (see Stoger E, et al. Molecular Breeding 9: 149-158, 2002, Fisher R, et al. Vaccine 21 (2003) 820-825, Ma J K, et al. Nat Rev Genet. 2003 October; 4(10):794-805, Schillberg S, et al. Vaccine 23 (2005) 1764-1769).

### Conjugate

The invention also relates to a conjugate comprising the mutant antibody fragment according to the invention, bonded to at least one, in particular one or two, molecules of interest, advantageously via the cysteine, and more particularly the thiol function of the cysteine, in position 128 of the heavy chain variable domain according to the IMGT nomenclature of the mutant antibody fragment.

### Molecule or Molecules of Interest Conjugated to the Mutant Antibody Fragment According to the Invention

Different types of molecules of interest can be conjugated to the mutant antibody fragment according to the invention, depending on the intended use of the conjugate.

The mutant antibody fragment according to the invention can, in particular, be conjugated to one or more molecules chosen from a molecule that is detectable or able to become detectable, an affinity molecule and a pharmacomodulating molecule.

In one embodiment, the mutant antibody fragment according to the invention can, in particular, be conjugated to one or more molecules that are detectable or able to become detectable, in particular in the context of diagnostic uses of the conjugate. The mutant antibody fragment according to the invention can, in particular, be conjugated to one (conjugate intended for use in a single type of imaging) or two (conjugate intended for use in several types of imaging, detecting different physical phenomena, bearing two detectable molecules or molecules able to become detectable of different type, or conjugate intended for use in a single type of imaging bearing two copies of the same molecule which is detectable or able to become detectable in order to increase the signal) molecules which are detectable or able to become detectable.

Each molecule that is detectable or able to become detectable is chosen according to the type of imaging for which the conjugate is intended. Examples of molecules that are detectable or able to become detectable that can be conjugated to the mutant antibody fragment according to the invention are:
a) a radioisotope (detectable molecule), a chelating agent (molecule able to become detectable, which becomes detectable after contact with a radiometal), or an affinity molecule (molecule able to become detectable, which becomes detectable after contact of the mutant antibody fragment conjugated to the affinity molecule with a radioisotope conjugated to a binding partner of the affinity molecule, or with a chelating agent conjugated to a binding partner of the affinity molecule and then a radiometal).

Such detectable molecules are useful primarily when the conjugate is intended for use in isotopic imaging.

The radioisotope can be covalently conjugated to the mutant antibody fragment according to the invention, or non-covalently by complexation with a chelating agent covalently conjugated to the mutant antibody fragment according to the invention.

Radioisotopes or radiometals that emit gamma rays directly (direct emission of a gamma photon when decaying) or indirectly (emission of a positron when decaying, which after annihilation with a surrounding electron leads to the emission of 2 gamma rays) are preferred because they can be detected by positron emission tomography (PET) or single-photon emission computed tomography (SPECT).

Some of the radioisotopes used in isotopic imaging are radiometals, generally conjugated indirectly to the probe used (here the mutant antibody fragment according to the invention), the probe comprising a chelating agent which binds the radiometal.

Examples of chelating agents include linear or cyclic chelating agents, such as DOTA, NOTA, deferoxamine B, and the derivatives thereof. Preferred derivatives of DOTA and NOTA are, for example, DOTAGA and (R)-NODAGA respectively.

Examples of radiometals that emit gamma rays directly or indirectly (via the emission of a positron, which after annihilation with a surrounding electron, leads to the emission of 2 gamma rays) include ^{99m}Tc, ¹¹¹In, ⁶⁴Cu, ⁶⁸Ga, ⁸⁹Zr, and ⁴⁴Sc. ^{99m}Tc, and ¹¹¹In are direct gamma-ray emitters detectable by single-photon emission computed tomography (SPECT), while ⁶⁴CU, ⁶⁸Ga, ⁸⁹Zr, and ⁴⁴Sc are indirect gamma-ray emitters detectable by positron emission tomography (PET).

The use of a chelating agent can be advantageous because it allows the conjugate to be stored ready for chelation and the radioisotope, which may have a very short half-life, to be added only at the last moment before use. In this case, complexation can be carried out just before use of the conjugate of the invention for an isotopic imaging application. Although conjugation to the chelating agent can be indirect via an affinity molecule and its binding partner, the mutant antibody fragment according to the invention is advantageously conjugated directly to a chelating agent.

Another radioisotope that can be used in PET imaging is ¹⁸F, which is a positron emitter (and therefore an indirect emitter of gamma photons) and which can, in particular, be introduced on so-called prosthetic groups, known to a person skilled in the art, which can be conjugated to the mutant antibody fragment according to the invention.
b) a fluorophore (detectable molecule) or an affinity molecule (molecule able to become detectable, the fluorophore is then detected after contact of the mutant antibody fragment conjugated to the affinity molecule with a fluorophore conjugated to a binding partner of the affinity molecule).

Fluorophores are useful when the conjugate is to be used for fluorescence imaging. As with radiometals in the case of radioisotopes, the fluorophore can be covalently conjugated to the mutant antibody fragment according to the invention, or non-covalently when it is conjugated beforehand to a binding partner of the affinity molecule which complexes with the affinity molecule covalently conjugated to the mutant antibody fragment according to the invention.

Examples of fluorophores that can be detected by fluorescence imaging and therefore conjugated to the mutant antibody fragment according to the invention, include compounds from the cyanine family (in particular cyanine 3, cyanine 5, cyanine 5.5, cyanine 7, cyanine 7.5, with or without water-solubilising groups, such as sulfones); compounds of the xanthene family (in particular compounds of the rhodamine family such as X-rhodamine, rhodamine B and compounds of the fluorescein family, with or without water-solubilising groups, such as sulfones); compounds of the coumarin family (in particular, hydroxycoumarin, aminocoumarin, methoxycoumarin, whether or not bearing water-solubilising groups, such as sulfones); and compounds of the boron dipyrromethene family (BODIPY, including azaBODIPY derivatives). The fluorophore is preferably selected from cyanine 5, cyanine 7, a rhodamine or an azaBODIPY.

Examples of (affinity molecule/binding partner) pairs include (biotin/avidin), (biotin/streptavidin), (avidin/biotin), and (streptavidin/biotin).
c) a chromophore (detectable molecule) or an affinity molecule (molecule able to become detectable, the chromophore is then detected after bringing the biotin-conjugated mutant antibody fragment into contact with a chromophore conjugated to a streptavidin molecule).

Chromophores are useful when the conjugate is intended for use in optical imaging by absorbance measurement. As with radiometals in the case of radioisotopes, the chromophore can be covalently conjugated to the mutant antibody fragment according to the invention, or non-covalently when it is conjugated beforehand to a binding partner of the affinity molecule which complexes with the affinity molecule covalently conjugated to the mutant antibody fragment according to the invention.

Examples of chromophores that may be conjugated to the mutant antibody fragment according to the invention for use in optical imaging include phenolphthalein, gentian violet or Congo Red.

Examples of (affinity molecule/binding partner) pairs include (biotin/avidin), (biotin/streptavidin), (avidin/biotin), and (streptavidin/biotin).

Thus, molecules that are detectable or able to become detectable may be selected from a fluorophore, a chromophore, an affinity molecule, a radioisotope and a chelating agent.

When the mutant antibody fragment according to the invention is conjugated to two molecules that are detectable or able to become detectable, it may in particular be conjugated to:
1) a radioisotope or chelating agent for use in isotope imaging and a fluorophore or affinity molecule for use in fluorescence imaging;
2) two molecules that are detectable or able to become detectable, of the same type and preferably identical, to increase the detected signal. In this embodiment, the molecule that is detectable or able to become detectable can be chosen from:
a) radioisotopes or chelating agents for use in isotope imaging, or
b) fluorophores or affinity molecules for use in fluorescence imaging.

The mutant antibody fragment according to the invention may additionally or alternatively be conjugated to an affinity molecule.

Such a conjugate can then be complexed with a detectable molecule or a molecule able to become detectable (for example, a chelating agent) capable of forming a complex with the affinity molecule which is conjugated to the mutant antibody fragment according to the invention.

Examples of affinity molecules include a biotin (capable, in particular, of forming a complex with a streptavidin previously conjugated to a fluorophore, a chromophore, a radioisotope or a chelating agent), an avidin (capable, in particular, of forming a complex with a biotin previously conjugated to a fluorophore, a chromophore, a radioisotope or a chelating agent), and a streptavidin (capable in particular of forming a complex with a biotin previously conjugated to a fluorophore, a chromophore, a radioisotope or a chelating agent).

Alternatively, the affinity molecule can be used to easily purify the mutant antibody fragment according to the invention. In this case, in addition to the affinity molecules mentioned in the previous paragraph, a hexahistidine peptide can also be used, enabling the fragment to be easily purified on a column comprising nickel (the binding partner to which the hexahistidine peptide complexes).

The affinity molecule can therefore be chosen from biotin, avidin, streptavidin and a hexahistidine peptide.

The mutant antibody fragment according to the invention may additionally or alternatively be conjugated to a pharmacomodulatory molecule. Such conjugation may enable the pharmacokinetics of the mutant antibody fragment according to the invention to be improved. Examples of pharmacomodulating molecules include linear or branched chains of poly(ethylene glycol), linear or branched chains of poly(glutamic acid), cholesterol, and molecules capable of binding to albumin (for example fatty acids, metal ions, bilirubin, a toxin, the p-iodophenyl butyryl group, the biphenyl group).

### Structure of the Conjugates

The conjugate according to the invention is bonded to at least one, in particular one or two, molecules of interest, advantageously via the cysteine, and more particularly the thiol function of the cysteine, in position 128 of the heavy chain variable domain according to the IMGT nomenclature of the mutant antibody fragment.

According to a first embodiment, the conjugate is bonded to two molecules of interest, advantageously via the cysteine, and more particularly the thiol function of the cysteine, in position 128 of the heavy chain variable domain according to the IMGT nomenclature of the mutant antibody fragment.

Advantageously, at least one of these molecules is a molecule that is detectable or is able to become detectable, and preferably the two molecules each independently represent a molecule that is detectable or is able to become detectable, such as a fluorophore, a chromophore, an affinity molecule, a radioisotope or a chelating agent. For example, one of these detectable molecules could be a fluorophore and the other could be a radioisotope or a chelating agent. For example, one of the molecules could be the chelating agent DOTA (allowing the chelation of a metallic radioisotope such as 68Ga) and the other a tetrasulfonated cyanine 7 (a fluorophore emitting in the near infrared).

Such a conjugate may correspond to the following formula (I):

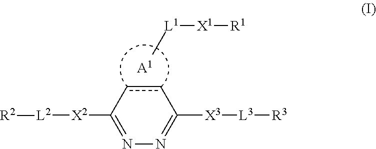

In which:
R¹ represents a mutant antibody fragment according to the invention bonded to X¹ via cysteine, and more particularly the thiol function of cysteine, in position 128 of the heavy chain variable domain according to the IMGT nomenclature;
X¹ represents

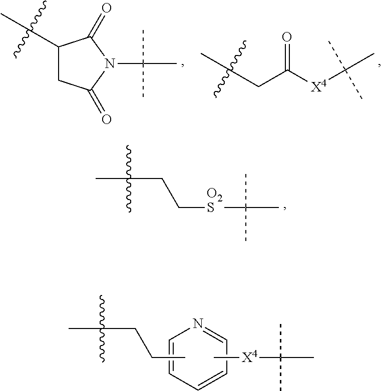

such as

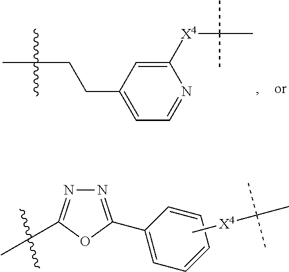

such as

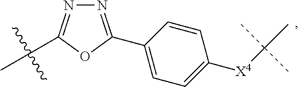

with X⁴ representing a single bond, O, S, or NR⁴, and R⁴ representing H or (C₁-C₆)alkyl, the wavy bond indicating the point of attachment to R¹ and the dashed bond indicating the point of attachment to L¹;
L¹, L² and L³ each independently represent a single bond or a spacer, the spacer being a (C₁-C₃₀)-alkyl chain, for example (C₁-C₂₀)-alkyl, optionally preceded and/or interrupted and/or followed by one or more units selected from the group consisting of aromatic, heteroaromatic, cycloalkane, cycloalkene, heterocycloalkane, heterocycloalkene, —O—, —S—, —NR⁵—, —C(O)—, —C(S)—, —C≡C—, and —C(R⁶)═C(R⁷)—, with R⁵ representing H or (C₁-C₆)alkyl, where R⁶ and R⁷ are each independently H or (C₁-C₆)alkyl, and the aromatic, heteroaromatic, cycloalkyl, cycloalkenyl, heterocycloalkyl and heterocycloalkenyl rings being optionally substituted by one or more (C₁-C₆)alkyl groups;
[symbol] represents a double bond or a single bond, preferably a double bond;
ring A¹ represents a cycloalkane, cycloalkene, heterocycloalkane or heterocycloalkene ring, optionally fused with one or more, in particular one or two, aromatic rings and optionally substituted with one or more groups chosen from (C₁-C₆)alkyl, halide and hydroxy;
X² and X³, which are identical or different, each represent 0, S or NR⁸, with R⁸ representing H or (C₁-C₆)alkyl, preferably H;
R² and R³, which are identical or different, preferably different, each represent a residue of a molecule of interest advantageously chosen from molecules that are detectable or able to become detectable, affinity molecules and pharmacomodulating molecules.

Preferably, X¹ represents

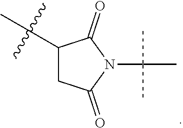

Preferably, L¹, L² and L³ each independently represent a spacer. According to a first embodiment, the spacer is a (C₁-C₃₀)-alkyl chain, in particular (C₁-C₂₀)-alkyl, such as (C₁-C₁₀)-alkyl or (C₁-C₆)-alkyl, and in particular an ethyl or hexyl group (e.g. n-hexyl). According to a second embodiment, the spacer is a PEG chain of formula —(CH₂CH₂O)ₓ— or —(CH₂CH₂O)ₓ—CH₂CH₂— with x an integer from 1 to 12, in particular from 2 to 8 and in particular 2, 4 or 8. According to a third embodiment, the spacer is a (C₁-C₃₀)-alkyl group, in particular (C₁-C₂₀)-alkyl, such as (C₁-C₁₀)-alkyl or (C₁-C₆)-alkyl, preceded or followed by a C(O) group such as a propanoyl group.

Preferably, at least one of X² and X³ represents S. Advantageously, X² and X³ are independently selected from S and NR⁸. Preferably, one of X² and X³ is S and the other is S or NR⁸. Advantageously, R⁸ is H.

Preferably, ring A¹ will be selected from

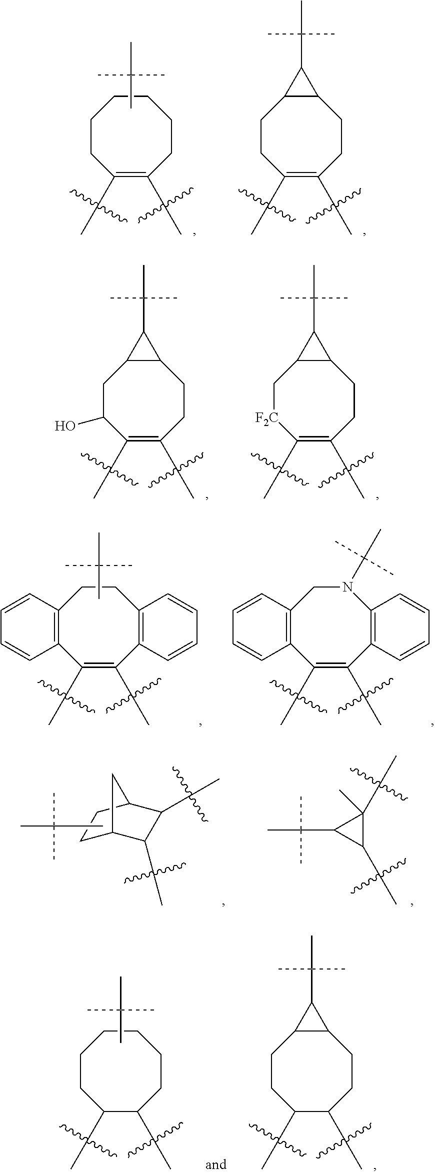

the dashed bond indicating the point of attachment to L¹ and the wavy bonds indicating the points of attachment to the carbon atoms bearing X² or X³.

Preferably, at least one of R² and R³ is a residue of a molecule that is detectable or able to become detectable, and in particular R² and R³ each independently represent a residue of a molecule that is detectable or able to become detectable, advantageously R² and R³ represent respectively a residue of a fluorophore and a residue of a radioisotope or of a chelating agent. In particular, R² and R³ can respectively represent a residue of a sulfonated Cyanine 7 and a residue of R-NODAGA (chelating agent).

According to a second embodiment, the conjugate is bonded to a single molecule of interest which is advantageously a molecule that is detectable or able to become detectable, such as a fluorophore, a chromophore, an affinity molecule, a radioisotope or a chelating agent; and this advantageously via the cysteine, and more particularly the thiol function of the cysteine, in position 128 of the heavy chain variable domain according to the IMGT nomenclature of the mutant antibody fragment.

Such a conjugate may correspond to the following formula (IIa) or (IIb):

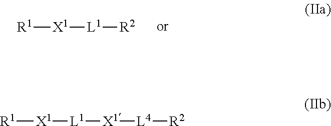

In which:
R¹ represents a mutant antibody fragment according to the invention bonded to X¹ via cysteine, and more particularly the thiol function of cysteine, in position 128 of the heavy chain variable domain according to the IMGT nomenclature;
X¹ represents

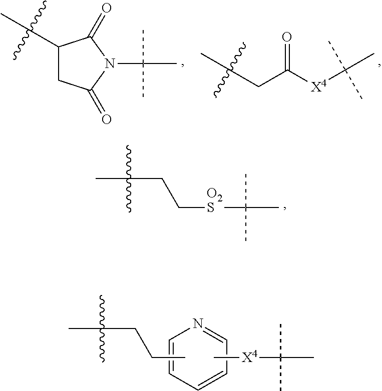

such as

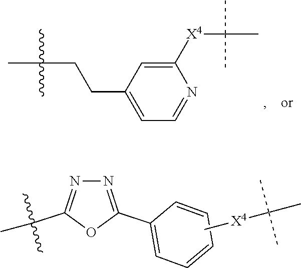

such as

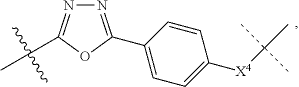

with X⁴ representing a single bond, O, S, or NR⁴, and R⁴ representing H or (C1-C₆)alkyl, the wavy bond indicating the point of attachment to R¹ and the dashed bond indicating the point of attachment to L¹;

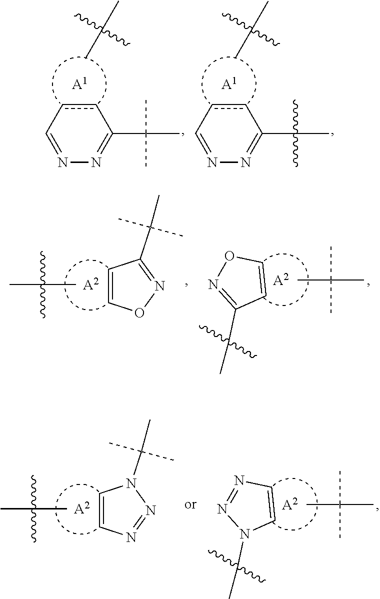

ring A¹ being as defined above and ring A² representing a cycloalkene or heterocycloalkene ring, optionally fused with one or more, in particular one or two, aromatic rings and optionally substituted with one or more groups selected from (C₁-C₆)alkyl, halide, and hydroxy, the wavy bond indicating the point of attachment to L⁴, and the dashed bond indicating the point of attachment to L¹;
L¹ and L⁴ each independently represent a single bond or a spacer, the spacer being a (C₁-C₃₀)-alkyl chain, for example (C₁-C₂₀)-alkyl, optionally preceded and/or interrupted and/or followed by one or more units selected from the group consisting of aromatic, heteroaromatic, cycloalkane, cycloalkene, heterocycloalkane, heterocycloalkene, —O—, —S—, —NR⁵—, —C(O)—, —C(S)— rings, —C≡C—, and —C(R⁶)═C(R⁷)—, with R⁵ representing H or (C₁-C₆)alkyl, where R⁶ and R⁷ are each independently H or (C₁-C₆)alkyl, and the aromatic, heteroaromatic, cycloalkyl, cycloalkenyl, heterocycloalkyl and heterocycloalkenyl rings being optionally substituted by one or more (C₁-C₆)alkyl groups;
R² represents a residue of a molecule of interest advantageously chosen from molecules that can be detected or are able to become detectable, affinity molecules and pharmacomodulating molecules.

Preferably, X¹ represents

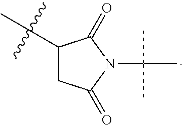

Preferably, L¹ and L⁴ each independently represent a spacer. According to a first embodiment, the spacer is a (C₁-C₃₀)-alkyl chain, in particular (C₁-C₂₀)-alkyl, such as (C₁-C₁₀)-alkyl or (C₁-C₆)-alkyl, and in particular an ethyl or hexyl group (e.g. n-hexyl). According to a second embodiment, the spacer is a PEG chain of formula —(CH₂CH₂O)ₓ— or —(CH₂CH₂O)ₓ—CH₂CH₂— with x an integer from 1 to 12, in particular from 2 to 8 and in particular 2, 4 or 8. According to a third embodiment, the spacer is a (C₁-C₃₀)-alkyl group, in particular (C₁-C₂₀)-alkyl, such as (C₁-C₁₀)-alkyl or (C₁-C₆)-alkyl, preceded or followed by a C(O) group such as a propanoyl group.

Preferably, X^{1′} represents

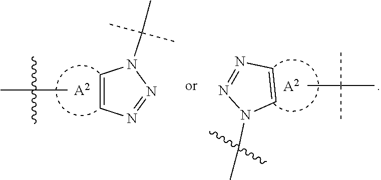

Preferably, ring A² will be selected from

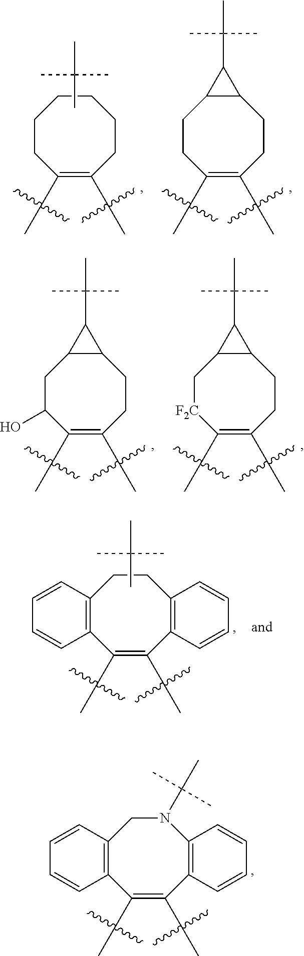

the dashed bond indicating the point of attachment to L¹ or L⁴ and the wavy bonds indicating the points of attachment to the N—N═N or O—N═N pattern.

Preferably, R¹ is a residue of a molecule that is detectable or able to become detectable, advantageously chosen from a fluorophore, a chromophore, an affinity molecule, a radioisotope and a chelating agent, and in particular from a fluorophore, a radioisotope and a chelating agent.

### Conjugation Methods and Synthesis Intermediates

When the mutant antibody fragment according to the invention comprises one or more disulfide bridges (in particular Fab, minibody, scFv, diabody, tribody and tetrabody fragment), the conjugates according to the invention can advantageously be prepared by a preparation method comprising the following steps in the order indicated:
a) reducing the disulfide bridge or bridges of the mutant antibody fragment, in particular in the presence of tris(2-carboxyethyl)phosphine (TCEP), to give a reduced mutant antibody fragment,
b) re-oxidising the disulfide bridge or bridges of the mutant antibody fragment, in particular in the presence of dehydroascorbic acid, to give a partially reduced mutant antibody fragment,
c) conjugating, with at least one molecule of interest, the thiol function of the cysteine in position 128 of the heavy chain variable domain according to the IMGT nomenclature of the partially reduced mutant antibody fragment, to give a conjugate according to the invention.

This method, comprising steps a) and b) as described herein prior to conjugation step c), enables highly specific conjugation at the cysteine, and more particularly the thiol function of the cysteine, in position 128 of the heavy chain variable domain according to the IMGT nomenclature of the mutant antibody fragment.
Step a) aims to reduce all the disulfide bridges of the mutant antibody fragment, both the disulfide bridges linking the heavy chains of the mutant antibody fragment (also known as inter-chain disulfide bridges) and the disulfide bridge formed with the thiol function of the cysteine in position 128.
Step b) aims to reoxidise only the inter-chain disulfide bridges, so as to obtain a partially reduced mutant antibody fragment, i.e. one in which only the thiol of the cysteine at position 128 is in reduced form. This thiol is then in the form of an SH group that can be used as a reactive chemical group to covalently conjugate at least one molecule of interest.
Step c) aims to conjugate the at least one molecule of interest to the SH function of the cysteine at position 128. To do this, the partially reduced mutant antibody fragment obtained in step b) will be coupled with a molecule bearing, on the one hand, at least one molecule of interest and, on the other hand, a chemical group capable of reacting with a thiol function SH.

Such a chemical group capable of reacting with an SH function may be, in particular,

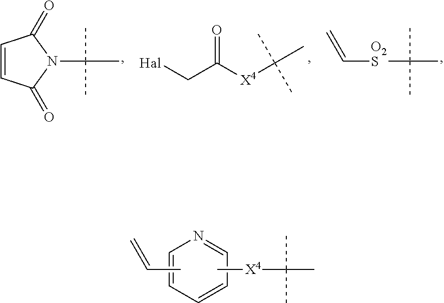

such as

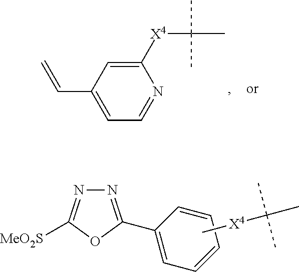

such as

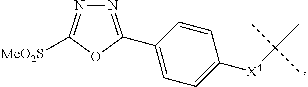

with HaI representing Br or I and X⁴ representing a single bond, O, S, or NR⁴, R⁴ representing H or (C₁-C₆)alkyl.

The method described above comprising steps a) and b) as described herein prior to step c) of conjugation is also preferred for antibody fragments, in particular the Fv, VHH and VNAR fragments, as these fragments are often isolated in the form of dimers or adducts with glutathione, cysteines, etc. Steps a) of reduction and b) of reoxidation before conjugation are therefore advantageously also used.

According to a first particular embodiment of the invention, the conjugate according to the invention is a conjugate of formula (I) as defined above and step c) can be carried out by coupling between the partially reduced mutant antibody fragment (corresponding to the molecule R₁H) and a compound of the following formula (III):

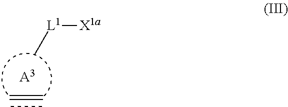

for which:
L¹ is as defined above,
ring A³ represents a cycloalkyne, cycloalkene, heterocycloalkyne or heterocycloalkene ring, optionally fused with one or more, in particular one or two, aromatic rings and optionally substituted with one or more groups chosen from (C₁-C₆)alkyl, halide and hydroxy, and
X^{1a} represents a chemical group that is reactive towards an SH function,
to give a compound of the following formula (IV):

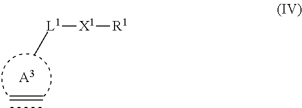

wherein R¹, X¹, L¹ and ring A³ are as defined above,
followed by coupling between the compound of formula (IV) and a compound of the following formula (V):

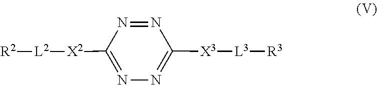

wherein R², R³, L², L³, X² and X³ are as defined above.

Preferably, ring A³ will be selected from

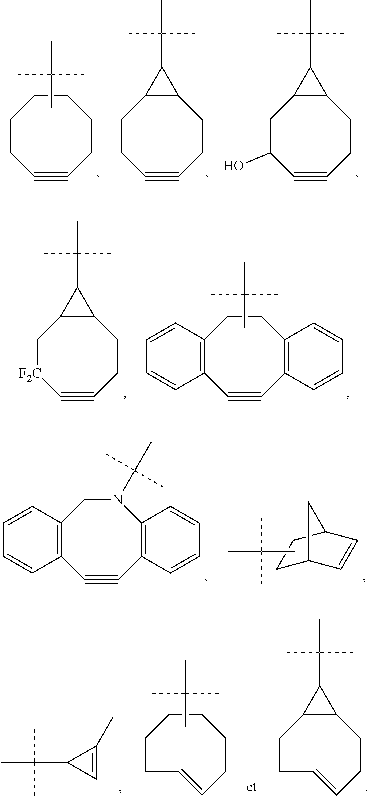

Preferably, X^{1a}, which represents a chemical group reactive towards an SH function, may be chosen from

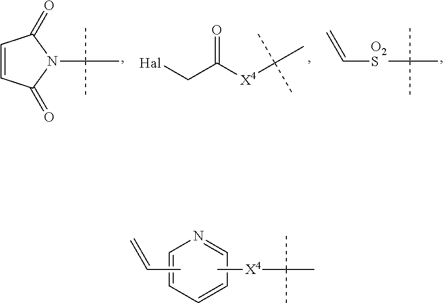

such as

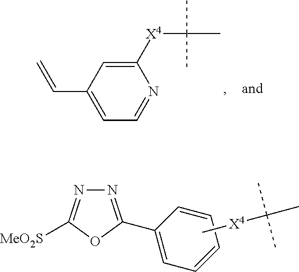

such as

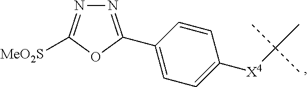

with HaI representing Br or I, X⁴ representing a single bond, O, S, or NR⁴, and R⁴ representing H or (C₁-C₆)alkyl. Reaction of X^{1a} with the SH function of cysteine at position 128 will form the group X¹ and covalently link the compound of formula (III) to the partially reduced mutant antibody fragment.

Preferably, this will be

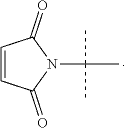

Such a reaction between the SH function of a cysteine and a chemical group reactive towards an SH function is well known in the art.

The A³ ring carried by the compound of formula (IV) thus formed can then be used to graft two molecules of interest via the reaction of this A³ ring with the tetrazine group of the compound of formula (V) which carries the two molecules of interest (R² and R³). This coupling reaction is well known to a person skilled in the art and is described in particular in WO2018/172543. The compound of formula (V) bearing the tetrazine group can also be prepared according to a method described in WO2018/172543.

According to a second particular embodiment of the invention, the conjugate is a conjugate of formula (IIa) or (IIb) as defined above.
Step c) will advantageously involve coupling of the partially reduced mutant antibody fragment (R¹H) with a compound bearing a chemical group reactive towards an SH function (X^{1a}). Such a coupling, reacting an SH thiol function with a chemical group reactive towards an SH function (Xia), is well known in the art.

The chemical group X^{1a} reactive towards an SH function may or may not be grafted directly onto a molecule of interest via a spacer L¹ (when L¹ represents a single bond). This makes it possible to prepare a conjugate of formula (IIa). In this case, step c) can be performed by coupling the partially reduced mutant antibody fragment (R¹H) with a compound of formula X^{1a}-L¹-R² wherein L¹, X^{1a} and R² are as defined above.

Instead, a different reactive chemical group (G²) may or may not be grafted to the molecule of interest via a spacer L⁴ (when L⁴ represents a single bond). In this case, the partially reduced mutant antibody fragment (R¹H) is conjugated to the molecule of interest (bearing a reactive group G²) using a bifunctional compound bearing:
at one end, an SH-reactive chemical group (X^{1a}) so that it can couple to the partially reduced mutant antibody fragment (R¹H), and
at the other end, a chemical group G¹ capable of reacting with G² so as to allow coupling with the molecule of interest.

This makes it possible to prepare a conjugate of formula (IIb).

According to a first aspect, step c) can be carried out by coupling the partially reduced mutant antibody fragment (R¹H) with a compound of formula X^{1a}-L¹-X^{1′}-L⁴-R² respectively, wherein L¹, X^{1a}, X^{1a}, L⁴ and R² are as defined above.

The compound of formula X^{1a}-L¹-X^{1′}—R² may then be prepared by coupling between a compound of formula X^{1a}-L¹-G¹ and a compound of formula G²-L⁴-R² wherein X^{1a}, L¹, L⁴ and R² are as defined above and G¹ and G² are different and each represents a reactive chemical group capable of reacting together to form a group X^{1′}.

The chemical groups G¹ and G² may be chosen according to Table 2 below, ring A³ being as defined above and ring A⁴ representing a cycloalkyne or heterocycloalkyne ring optionally fused with one or more, in particular one or two, aromatic rings and optionally substituted by one or more groups chosen from (C₁-C₆)alkyl, halide and hydroxyl:

TABLE 2

| 1^{st} reactive group | 2^{nd} reactive group (G¹ or G²) |
|---|---|
| (G¹ or G²) | capable of reacting with the 1^{st} reactive group |
| 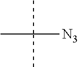 | 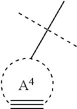 |
| 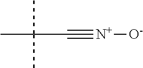 | 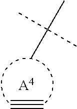 |
| 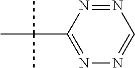 | 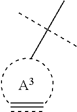 |

According to a second aspect, step c) can be performed by coupling the partially reduced mutant antibody fragment (R¹H) with a compound of formula X^{1a}-L¹-G¹ wherein L¹, G¹ and X^{1a} are as defined above, to give a conjugate of formula R¹—X¹-L¹-G¹, followed by coupling between the conjugate of formula R¹—X¹-L¹-G¹ and a compound of formula G²-R² wherein G² and R² are as defined above.

The present invention therefore also relates to intermediate conjugates that are useful as synthesis intermediates during the preparation of the conjugates according to the invention.

Another object of the present invention is an intermediate conjugate comprising the mutant antibody fragment according to the invention, bonded to a reactive chemical group via the cysteine, and more particularly the thiol function of the cysteine, in position 128 of the heavy chain variable domain according to the IMGT nomenclature of the mutant antibody fragment.

Preferably, the reactive chemical group is selected from

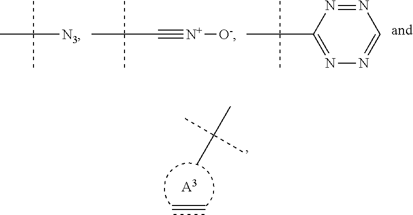

preferably

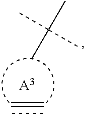

such as

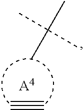

the rings A³ and A⁴ being as defined above.

Preferably, ring A³ will be chosen from

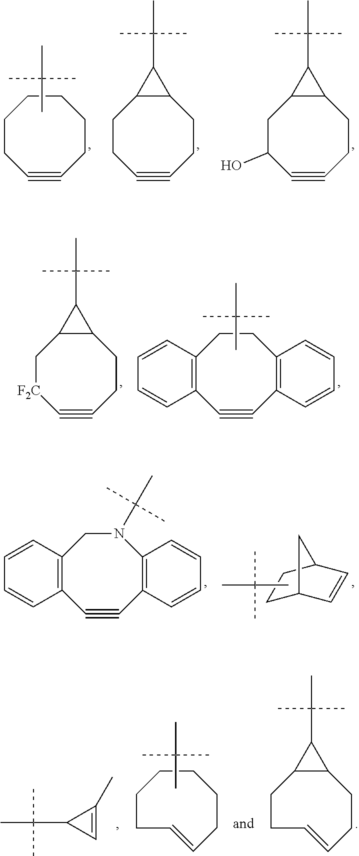

Preferably, ring A³ will be a ring A⁴ which will advantageously be chosen from

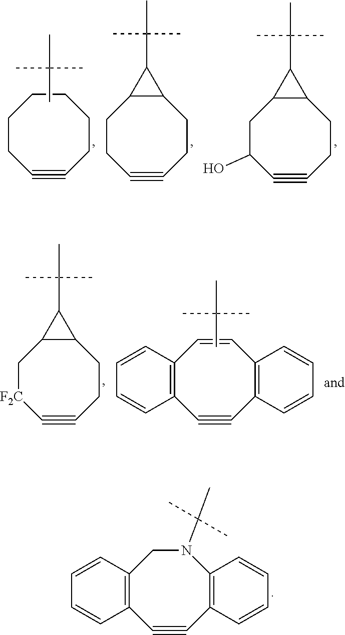

According to a particular embodiment, the intermediate conjugate according to the invention has the formula R¹—X¹-L¹-Z wherein R¹, X¹ and L¹ are as defined above, and Z represents a reactive chemical group as defined above.

### Uses of the Conjugate

Depending on the one or more molecules conjugated to the mutant antibody fragment according to the invention at the cysteine in position 128 of the heavy chain according to the IMGT nomenclature, the conjugate according to the invention can be used in various in vivo applications.

In particular, the present invention also relates to the conjugate according to the invention, for use in:
a) a method for diagnosing a tumour by imaging,
b) a method for surgical treatment by tumour resection guided by fluorescence or isotopic imaging probe,
c) a method for monitoring the effectiveness of an anti-tumour treatment by imaging; or
d) any combination of methods a), b) and c) (such as a) and b); a) and c); b) and c); or a), b) and c)).

### Method for Diagnosing a Tumour by Imaging

The invention therefore concerns the conjugate according to the invention, for use in a method for diagnosing a tumour by imaging. The present invention also relates to the use of a conjugate according to the invention in a method for diagnosing a tumour by imaging.

The present invention also relates to a method for detecting tumour cells in a subject by imaging, comprising:
a) administering, to a subject suspected of suffering from a tumour in an organ, a conjugate according to the invention, the antibody fragment of which binds specifically to an antigen of the suspected tumour,
b) obtaining, by imaging, one or more images of the organ suspected of containing a tumour, and
c) detecting the presence or absence of conjugate-labelled tumour cells in the one or more images obtained in step b).

The present invention also relates to a method for determining tumour burden in a tumour patient by imaging, comprising:
a) administering, to a patient suffering from a tumour, a conjugate according to the invention, the antibody fragment of which binds specifically to an antigen of the patient's tumour,
b) obtaining, by imaging, one or more images of one or more organs of the patient, and
c) determining the patient's tumour burden from the one or more images obtained in step b).

In this application, the one or more molecules conjugated to the mutant antibody fragment according to the invention at the cysteine in position 128 of the heavy chain according to the IMGT nomenclature is advantageously chosen from molecules that are detectable or able to become detectable.

The one or more molecules that are detectable or are able to become detectable will be chosen according to the type or types of imaging envisaged.

For use in a method for diagnosing a tumour by isotopic imaging, the mutant antibody fragment according to the invention is advantageously conjugated, at the cysteine in position 128 of the heavy chain according to the IMGT nomenclature, to a radioisotope or a chelating agent (a complex with a radiometal is then formed, just before administration to the patient). In particular, for positron emission tomography (PET) or single-photon emission computed tomography (SPECT), the mutant antibody fragment according to the invention is advantageously conjugated, at the cysteine in position 128 of the heavy chain according to the IMGT nomenclature, to a radioisotope emitting gamma rays directly or indirectly (in particular ¹⁸F) or to a chelating agent (a complex with a radiometal emitting gamma rays directly or indirectly, in particular selected from ^{99m}Tc, ¹¹¹In, ⁶⁴Cu, ⁶⁸Ga, ⁸⁹Zr, and ⁴⁴Sc, is then formed just before administration to the patient). For Cerenkov luminescence imaging (CLI), the mutant antibody fragment according to the invention is advantageously conjugated, at the cysteine in position 128 of the heavy chain according to the IMGT nomenclature to ⁹⁰Y, ⁶⁸Ga, ²²⁵Ac.

For use in a method for diagnosing a tumour by optical imaging, the mutant antibody fragment according to the invention is advantageously conjugated, at the cysteine in position 128 of the heavy chain according to the IMGT nomenclature, to a chromophore or an affinity molecule (a complex with a chromophore conjugated to a binding partner of the affinity molecule is then formed before administration to the patient, the (affinity molecule/binding partner) pair advantageously being chosen from (biotin/avidin), (biotin/streptavidin), and (avidin/biotin), (streptavidin/biotin)).

For use in a method for diagnosing a tumour by fluorescence imaging, the mutant antibody fragment according to the invention is advantageously conjugated, at the cysteine in position 128 of the heavy chain according to the IMGT nomenclature, to a fluorophore or an affinity molecule (a complex with a fluorophore conjugated to a binding partner of the affinity molecule is then formed before administration to the patient, the (affinity molecule/binding partner) pair advantageously being chosen from (biotin/avidin), (biotin/streptavidin), (avidin/biotin) and (streptavidin/biotin)).

The mutant antibody fragment according to the invention can also be conjugated, at the cysteine in position 128 of the heavy chain according to the IMGT nomenclature, to two different detectable molecules or molecules able to become detectable, enabling it to be used in two different types of imaging.

For example, the mutant antibody fragment according to the invention can be conjugated, at the cysteine in position 128 of the heavy chain according to the IMGT nomenclature, to two molecules that are detectable or able to become detectable by different imaging techniques, and in particular to:
a1) a radioisotope emitting gamma rays directly or indirectly, or a chelating agent for use in a method for diagnosing a tumour by isotopic imaging (in particular by positron emission tomography (PET) or single-photon emission computed tomography (SPECT)) and a fluorophore for use in a method for diagnosing a tumour by fluorescence imaging;
a2) a radioisotope emitting gamma rays directly or indirectly, or a chelating agent for use in a method for diagnosing a tumour by isotopic imaging (in particular by positron emission tomography (PET) or by single-photon emission computed tomography (SPECT)) and an affinity molecule (a complex with a fluorophore conjugated to a binding partner of the affinity molecule is then formed before administration to the patient, the (affinity molecule/binding partner) pair being advantageously selected from (biotin/avidin), (biotin/streptavidin), (avidin/biotin), and (streptavidin/biotin)) for use thereof in a method for diagnosing a tumour by fluorescence imaging;
b1) a radioisotope which emits gamma rays directly or indirectly, or chelating agent for use in a method for diagnosing a tumour by isotope imaging (in particular by positron emission tomography (PET) or single-photon emission computed tomography (SPECT)) and a chromophore for use in a method for diagnosing a tumour by optical imaging;
b2) a radioisotope which emits gamma rays directly or indirectly, or a chelating agent for use in a method for diagnosing a tumour by isotopic imaging (in particular by positron emission tomography (PET) or by single-photon emission computed tomography (SPECT)) and an affinity molecule (a complex with a chromophore conjugated to a binding partner of the affinity molecule is then formed before administration to the patient, the (affinity molecule/binding partner) pair being advantageously selected from (biotin/avidin), (biotin/streptavidin), (avidin/biotin), and (streptavidin/biotin)) for use in a method for diagnosing a tumour by optical imaging;
c1) a fluorophore or an affinity molecule (a complex with a fluorophore conjugated to a binding partner of the affinity molecule is then formed before administration to the patient, the (affinity molecule/binding partner) pair advantageously being chosen from (biotin/avidin), (biotin/streptavidin), (avidin/biotin), and (streptavidin/biotin)) for use in a method for diagnosing a tumour by fluorescence imaging, and a chromophore for use thereof in a method for diagnosing a tumour by optical imaging; or
c2) a fluorophore for use in a method for diagnosing a tumour by fluorescence imaging and an affinity molecule (a complex with a fluorophore conjugated to a binding partner of the affinity molecule is then formed before administration to the patient, the (affinity molecule/binding partner) pair being advantageously selected from (biotin/avidin), (biotin/streptavidin), (avidin/biotin), and (streptavidin/biotin)) for use in a method for diagnosing a tumour by optical imaging.

Among the conjugates with two molecules detectable or able to become detected by different imaging techniques described above, conjugates a1 and b1) are preferred.

Alternatively, the mutant antibody fragment according to the invention can be conjugated, at the cysteine in position 128 of the heavy chain according to the IMGT nomenclature, to two molecules that are detectable or able to become detectable by the same imaging technique (this making it possible to increase the signal and therefore the detection sensitivity), and in particular to:
a) two radioisotopes which emit gamma rays directly or indirectly (different or preferably identical), or two chelating agents (different or preferably identical) for use in a method for diagnosing a tumour by isotopic imaging (in particular by positron emission tomography (PET) or single-photon emission computed tomography (SPECT)),
b) two fluorophores (different or preferably identical) for use thereof in a method for diagnosing a tumour by fluorescence imaging,
c) two chromophores (different or preferably identical) for use thereof in a method for diagnosing a tumour by optical imaging, or
d) two affinity molecules (different or preferably identical) for use thereof in a method for diagnosing a tumour by optical or fluorescence imaging.

Among the conjugates with two molecules that can be detected or are able to become detected by the same imaging technique described above, conjugates a) (two radioisotopes which emit gamma rays directly or indirectly (different or preferably identical) or two chelating agents, (different or preferably identical) and b) (two fluorophores, different or preferably identical) are preferred.

Alternatively, the mutant antibody fragment according to the invention can also be conjugated, at the cysteine in position 128 of the heavy chain according to the IMGT nomenclature, to a molecule that is detectable or able to become detectable and a pharmacomodulating molecule, the latter making it possible to modify the pharmacokinetic properties of the diagnostic conjugate.

In this embodiment, the mutant antibody fragment according to the invention can also be conjugated, at the cysteine in position 128 of the heavy chain according to the IMGT nomenclature, to:
a) a pharmacomodulating molecule and a radioisotope which emits gamma rays directly or indirectly, or a chelating agent for use in a method for diagnosing a tumour by isotopic imaging (in particular by positron emission tomography (PET) or single-photon emission computed tomography (SPECT));
b) a pharmacomodulating molecule and a fluorophore for use in a method for diagnosing a tumour by fluorescence imaging;
c) a pharmacomodulatory molecule and a chromophore for use in a method for diagnosing a tumour by optical imaging;
d) a pharmacomodulating molecule and an affinity molecule (a complex with a fluorophore conjugated to a binding partner of the affinity molecule is then formed before administration to the patient, the (affinity molecule/binding partner) pair being advantageously selected from (biotin/avidin), (biotin/streptavidin), (avidin/biotin), and (streptavidin/biotin)) for use in a method for diagnosing a tumour by optical or fluorescence imaging.

Among the conjugates with a molecule that is detectable or able to become detectable and a pharmacomodulating molecule described above, conjugates a) (a pharmacomodulating molecule and a radioisotope that emits gamma rays directly or indirectly or a chelating agent) and b) (a pharmacomodulating molecule and a fluorophore) are preferred.

### Method of Surgical Treatment by Tumour Resection Guided by Fluorescence or Nuclear Imaging Probe

The invention also relates to the conjugate according to the invention, for use in a method for surgical treatment by tumour resection guided by fluorescence or isotopic imaging probe.

The present invention also relates to a method for surgical treatment by tumour resection guided by fluorescence or by isotope imaging probe, comprising:
a) administering, to a patient, a conjugate according to the invention, the antibody fragment of which binds specifically to an antigen of the patient's tumour,
b) obtaining real-time imaging images of the conjugate-Labelled tumour, and surgical resection of the entire tumour using the real-time images obtained.

In this application, the mutant antibody fragment according to the invention is conjugated, at the cysteine in position 128 of the heavy chain according to the IMGT nomenclature, to:
a1) a fluorophore for use in a method for surgical treatment by tumour resection guided by fluorescence;
a2) an affinity molecule (a complex with a fluorophore conjugated to a binding partner of the affinity molecule is then formed before administration to the patient, the (affinity molecule/binding partner) pair advantageously being chosen from (biotin/avidin), (biotin/streptavidin), (avidin/biotin), and (streptavidin/biotin)) for use in a method for surgical treatment by tumour resection guided by fluorescence imaging;
b1) a chelating agent (in this case, a complex with a radiometal, which is a direct or indirect emitter of gamma rays, is formed just before administration to the patient) for its use in a method for surgical treatment by tumour resection guided by isotopic imaging probe, in particular by single-photon emission computed tomography (SPECT) imaging probe, in which case the radiometal is a direct emitter of gamma rays, such as ^{99m}Tc and ¹¹¹In) or by positron emission tomography (PET), in which case the radiometal is an indirect emitter of gamma rays, such as ⁶⁴Cu, ⁶⁸Ga, ⁸⁹Zr, and ⁴⁴Sc), with a preference for use in a tumour resection surgical treatment method guided by single-photon emission computed tomography (SPECT), where the radiometal is a direct emitter of gamma rays, such as ^{99m}Tc and ¹¹¹In);
b2) a radioisotope which emits gamma rays directly or indirectly (e.g. ¹⁸F), for use in a method for surgical treatment by tumour resection guided by isotope imaging probe;
c1) a molecule defined in a1) and a molecule defined in b), thereby enabling its use in a method for surgical treatment tumour resection guided by fluorescence or by isotope imaging probe; or
c2) a molecule defined in a2) and a molecule defined in b), thus enabling it to be used in a method for surgical treatment by tumour resection guided by fluorescence or by isotopic imaging probe.

In this method, the conjugates a1), b1) and c1) are preferred.

### Imaging Method for Monitoring the Efficacy of Anti-Tumour Treatment

The invention also relates to the conjugate according to the invention, for use in a method for monitoring, by imaging, the efficacy of an anti-tumour treatment.

The present invention also relates to a method for treating a cancer patient, comprising:
a) determining the patient's tumour burden by the method for determining the tumour burden according to the invention, before or at the same time as the start of administration of the anti-tumour treatment,
b) administering the anti-tumour treatment to the patient,
c) determining the patient's tumour burden by the method for determining the tumour burden according to the invention, after the start of administration of the anti-tumour treatment,
d) comparing the tumour burdens determined in steps a) and c), and
e) administering an anti-tumour treatment to the patient, where:
   if the tumour burden determined in step c) is less than or the same as that determined in step a), then the anti-tumour treatment administered in step e) is the same as in step b);
   if the tumour burden determined in step c) is greater than that determined in step a), then the anti-tumour treatment administered in step e) is different from that administered in step b).

In this application, the one or more molecules conjugated to the mutant antibody fragment according to the invention at the cysteine in position 128 of the heavy chain according to the IMGT nomenclature is advantageously chosen from molecules that are detectable or able to become detectable.

The molecule that is detectable or is able to become detectable will be chosen as a function of the type or types of imaging envisaged, as described above in the section concerning the use of the conjugate according to the invention in a method for diagnosing a tumour by imaging.

To determine the efficacy of an anti-tumour treatment, the conjugate according to the invention is advantageously used:
a) before or at the same time as the anti-tumour treatment is started, and
b) at least once after the start of administration of the anti-tumour treatment,
and the tumour burden detected after the start of administration of the anti-tumour treatment is compared with that detected before or at the same time as the start of administration of the anti-tumour treatment.

If the tumour burden has decreased or remains stable after the start of administration of the anti-tumour treatment, then the anti-tumour treatment is considered effective. If the tumour burden has increased after the start of administration of the anti-tumour treatment, then the anti-tumour treatment is considered ineffective.

The present invention is now illustrated by the examples below.

### EXAMPLES

### Example 1: Comparison of the Conservation of Residues in Positions 5, 24 and 128 of the Heavy Chain According to the IMGT Nomenclature

Junutula J R et al. have shown, for an Hu4D5 Fab (Fab of a human anti-HER2 antibody), that substitution of positions 5 and 23 of the heavy chain (according to a sequential numbering, each position number corresponding to the number of the amino acid in the amino acid sequence) by a cysteine allows good reactivity of the thiol group of the cysteine (see Table 2 of Junutula J R et al. Rapid identification of reactive cysteine residues for site-specific labeling of antibody-Fabs, Journal of Immunological Methods, Volume 332, Issues 1-2, 2008, Pages 41-52, https://doi.org/10.1016/j.jim.2007.12.011). These positions correspond to positions 5 and 24 according to the IMGT nomenclature. In Fab Hu4D5, the amino acids, at positions 5 and 24 of the heavy chain according to the IMGT nomenclature, are a valine (V) and an alanine (A) respectively.

The conservation of amino acids present in murine or human antibodies at positions 5, 24 and 128 of the heavy chain according to the IMGT nomenclature was analysed.

### Materials and Methods

The heavy chain of an antibody is generated by recombination between 3 gene segments called V, D and J.

Positions 5 and 24 of the heavy chain according to the IMGT nomenclature are located in segment V. Consequently, in order to analyse their conservation within murine and human antibodies, alignments of murine V segments and human V segments were obtained from the IMGT database.

Within these alignments, the various residues present in position 5 or 24 of the heavy chain according to the IMGT nomenclature were identified and their frequency (number of occurrences/number of V segments) determined.

Position 128 of the heavy chain according to the IMGT nomenclature is located in segment J. Consequently, in order to analyse its conservation within murine and human antibodies, alignments of murine J segments and human J segments were obtained from the IMGT database.

Within these alignments, the various residues present in position 128 of the heavy chain according to the IMGT nomenclature were identified and their frequency (number of occurrences/number of V segments) determined.

### Results

The results concerning position 5 of the heavy chain according to the IMGT nomenclature are presented in Table 3 below, and show that this position is poorly conserved and varies significantly depending on which V segment is used in the VH domain.

TABLE 3

| | Murine V segments | Human V segments |
|---|---|---|
| Amino acids in position 5 of | Q (60%), V (17%), K (17%), L | V (63%), Q (19%), I (6%), |
| the heavy chain (IMGT) | (2%), E (2%), P (0.7%), H | L (5%), K (4%), G (1%), |
| | (0.4%), N (0.4%), D (0.4%) M | R (1%), T (1%) |
| | (0.4%) | |

Thus, a large number of different amino acids may be present at this position, depending on the V segment used in the VH domain of the original antibody fragment, some of which have quite different properties (for example, valine V is one of the amino acids with a slightly hydrophobic uncharged side chain, while arginine R and lysine K are amino acids with a positively charged side chain, and glutamine Q and threonine T are amino acids with an uncharged polar side chain). Moreover, arginine R, lysine K and glutamine Q have significantly longer side chains than valine. While replacing a valine (V) with a cysteine results in good reactivity of the cysteine thiol, it is not clear that replacing an arginine R, a lysine K, a glutamine Q or a threonine T with a cysteine would lead to the same result.

Moreover, position 5 of the heavy chain according to the IMGT nomenclature is not suitable for introducing a substitution, as positions 6 and 7, located immediately next to it, are known to play a crucial role in the correct folding of the antibody chain. Introducing a cysteine C at this position could therefore disrupt the correct folding of the heavy chain (Honegger, A., Pluckthun, A., 2001. The Influence of the Buried Glutamine or Glutamate Residue in Position 6 on the Structure of Immunoglobulin Variable Domains. Journal of Molecular Biology 309, 687-699. https://doi.org/10.1006/jmbi.2001.4664; Jung S, Spinelli S, Schimmele B, Honegger A, Pugliese L, Cambillau C, et al. The Importance of Framework Residues H6, H7 and H10 in Antibody Heavy Chains: Experimental Evidence for a New Structural Subclassification of Antibody VH Domains. Journal of Molecular Biology 2001; 309:701-16). Consequently, replacing the residue in position 5 with a cysteine, and then conjugating this to a chemical group, could generate conformational instability in the variable region. Position 5 of the heavy chain according to the IMGT nomenclature is therefore not suitable for a general method of substituting this position with a cysteine to enable site-specific conjugation.

The results concerning position 24 of the heavy chain according to the IMGT nomenclature are presented in Table 4 below, and also show that this position is poorly conserved and varies significantly depending on which V segment is used in the VH domain.

TABLE 4

| | Murine V segments | Human V segments |
|---|---|---|
| Amino acids in position 24 of | K (48%), A (16%), V (15%), S | A (68%), K (22%), T (13% 6%), |
| the heavy chain (IMGT) and | (8%), T (7.6%), E (1.8%), Q | V (5%), S (1%) |
| their frequency (%) | (1.3%), M (1.3%), P (0.4%), D | |
| | (0.4%), I (0.4%) | |

Thus, a certain number of distinct amino acids are used at this position depending on the V segment used in the VH domain of the original antibody fragment, some of which have quite different properties (for example, valine V and alanine A are among the amino acids with a slightly hydrophobic uncharged side chain, while lysine K is among the amino acids with a positively charged side chain, and threonine T is among the amino acids with an uncharged polar side chain). Moreover, lysine K has a significantly longer side chain than alanine. While replacing an alanine (A) with a cysteine results in good reactivity of the cysteine thiol, it is not clear that replacing a lysine K or a threonine T with a cysteine would lead to the same result.

Moreover, position 24 of the heavy chain according to the IMGT nomenclature is not suitable for introducing a substitution, as it plays a crucial role in the correct folding of the antibody chain, being located just next to the cysteine in IMGT position 23 which forms the S—S bridge between the IMGT position of the C23 and C104a. Introducing a C at position 24 could therefore disrupt the correct folding and stabilisation of the heavy chain. The proximity of CDR1-H (which starts only 3 amino acids away) could also interfere with the interaction with the antigen once the chemical group has been conjugated at this position.

Position 24 of the heavy chain according to the IMGT nomenclature is therefore not suitable for a general method of substituting this position with a cysteine to enable site-specific conjugation.

Finally, the results concerning position 128 of the heavy chain according to the IMGT nomenclature are presented in Table 5 below, and show on the contrary that this position is highly conserved, with a single amino acid (S) being used by all human J segments, and only two distinct amino acids (S or A), one of which is common to that of human J segments, being used by mouse J segments.

TABLE 5

| | Murine V segments | Human V segments |
|---|---|---|
| Amino acids in | S (75%), A (25%) | S (100%) |
| IMGT position | | |
| 128 of the heavy chain | | |

A single amino acid (S) is present at IMGT position 128 of the heavy chain in human J segments, and the majority of amino acids present at IMG position 128 of the heavy chain in murine J segments are also serine, the only other amino acid used at this position being alanine. Moreover, serine and valine have in common that they are amino acids with a short side chain, and therefore with similar steric hindrance, which makes it plausible that what works by replacing an alanine at IMGT position 128 of the heavy chain with a cysteine will also work by replacing a serine at IMGT position 128 of the heavy chain with a cysteine.

Moreover, IMGT position 128 of the heavy chain corresponds to the last amino acid of the VH domain and is therefore not involved in antigen recognition or heavy chain folding.

For all these reasons, IMGT position 128 of the heavy chain is much more suitable than IMGT positions 5 and 24 of the heavy chain for a general method of substituting this position with a cysteine to allow site-specific conjugation.

### Conclusion

In summary, IMGT positions 5 and 24 of the heavy chain are poorly conserved and involved in the correct folding of the heavy chain (IMGT position 24 is also close to CDR1-H and also close to the cysteine in position 23 and their replacement by a cysteine could therefore interfere with the canonical disulfide bridge between the cysteines in positions 23 and 104. These positions are therefore not suitable for a general method for substituting this position with a cysteine to enable site-specific conjugation.

In contrast, IMGT position 128 of the heavy chain, the last amino acid before the constant region, is highly conserved and is not involved in either antigen recognition or heavy chain folding. It therefore lends itself particularly well to a general method for substituting this position with a cysteine to enable site-specific conjugation.

Moreover, its position in the VH domain means that it can be used for any type of antibody fragment of interest, as long as it comprises a VH domain. This is not the case for IMGT position 129 identified in Junutula J R et al. as the best position of the Hu4D5 Fab heavy chain for site-specific conjugation (Junutula J R et al. Rapid identification of reactive cysteine residues for site-specific labeling of antibody-Fabs, Journal of Immunological Methods, Volume 332, Issues 1-2, 2008, Pages 41-52, https://doi.org/10.1016/j.jim.2007.12.011). This position is in fact the first amino acid in the constant region. Thus, no fragment without a constant region (for example an scFv fragment, an Fv fragment, a diabody, a tribody, or a tetrabody) can be conjugated using this position.

### Example 2: Preparation of a Mutant Fab of the Chimeric Antibody xiRA63 with a Cysteine in Position 128 (IMGT Nomenclature) of the Heavy Chain and Random or Site-Specific Grafting onto a Tetrazine Already Bifunctionalised by a Zirconium-89 Chelating Agent (DFO) and a Fluorophore (IRDye800)

### Materials and Methods

### Production of ThioFab-xiRA63:

The heavy and light chains of Thiofab-xiRA63 or Fab-xiRA63 were cloned into the eukaryotic expression vector pTT5 (Durocher et al., 2002). Plasmids were co-transfected into ExpiCHO-S cells (ThermoFisher) according to the manufacturer's “ExpiCHO™ Expression System User Guide” (MAN0014337 ThermoFisher Scientific). Day 12 post-transfection, the supernatant was clarified by centrifugation at 4000-5000×g for 30 min at 4° C. and filtered through a 0.22 pm filter. ThioFab xiRA63 was then purified by affinity on a HiTrap KappaSelect column (GE Healthcare). After elution, antibody solutions were dialysed with the Slide-A-Lyzer™ G2 dialysis cassette (Thermoscientific) with a cut-off of 10 kDa in 1 litre of PBS.

### Conjugation of ThioFab-DFO-IRDye800

ThioFab xiRA63 bearing a cysteine was reduced in the presence of 15 equivalents of TCEP (20 mM, PBS) at 37° C. for 1 hour. After purification by ultrafiltration (Amicon 10 kDa cut-off), 20 equivalents of dehydroascorbic acid (dhAA, 50 mM, carbonate-bicarbonate buffer, pH 9) were added and the reaction medium was stirred at 25° C. for 1 hour to specifically reoxidise the disulfide bridges. Excess dhAA was removed by ultrafiltration and 20 equivalents of TCO-PEG₃-maleimide (15 mM, DMSO) were added before stirring for 2 hours at 25° C. Purification was then carried out by centrifugation. Finally, 15 equivalents of DFO-Tz-IRDye800 (30 mM, DMSO) were added and the IEDDA reaction proceeded overnight at 25° C. The ThioFab conjugate was then purified by steric exclusion chromatography (Superdex 75 10/300 GL).

Analysis of the [⁸⁹Zr]-ThioFab-xiRA63-DFO fragment by flow cytometry:

The protocol used was that of of Herbet et al. 2018 (Herbet A. et al. Antibodies Targeting Human Endothelin-1 Receptors Reveal Different Conformational States in Cancer Cells. Physiol. Res. 67 (Suppl. 1): 5257-5264, 2018. https://doi.org/10.33549/physiolres.933848). Two cell lines were used to assess the apparent affinity of the different antibody formats: Chinese Hamster Ovary Wild-Type (CHO-WT) and Chinese Hamster Ovary stably transfected with a eukaryotic expression vector encoding human ETA (CHO-ETA). Cells were resuspended in saturation buffer (1×PBS, 5% normal goat serum, 0.1% bovine serum albumin) and 3.10⁵ live cells were seeded in each tube. Apparent affinity was determined with a dilution range of 0.1 nM to 500 nM for ThioFab-xiRA63, ThioFab-xiRA63-DFO-IRDye800 and Fab-xiRA63 and Fab-xiRA63-DFO-IRDye800 fragments. The samples were incubated at 4° C. overnight. The following day, the system was revealed by a secondary goat anti-human IgG antibody Fd (CliniSciences 2046-02) conjugated to a fluorescent molecule, FITC, diluted 500^{th} and incubated for 4 hours at 4° C. The samples were washed three times with PBS between each step. Sample fluorescence was measured at a wavelength of 488 nm using the FACSCalibur flow cytometer (BD Biosciences, San Jose, CA, USA) and expressed as a percentage of the mean fluorescence intensity per sample. The “one site specific binding” function in GraphPad was applied to obtain the binding curve, Bmax and apparent Kd±SEM.

### Results

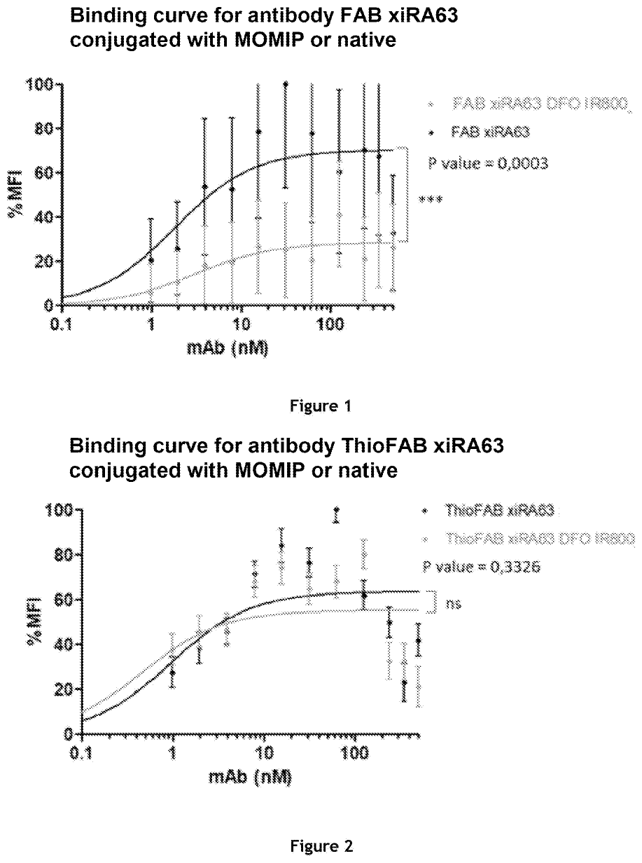

FIG. 1 shows antigen binding curves comparing the non-mutated Fab fragment of the xiRA63 antibody and a conjugate obtained by random grafting of a bifunctionalised tetrazine with a Zirconium-89 chelating agent (DFO) and a fluorophore (IRDye800).

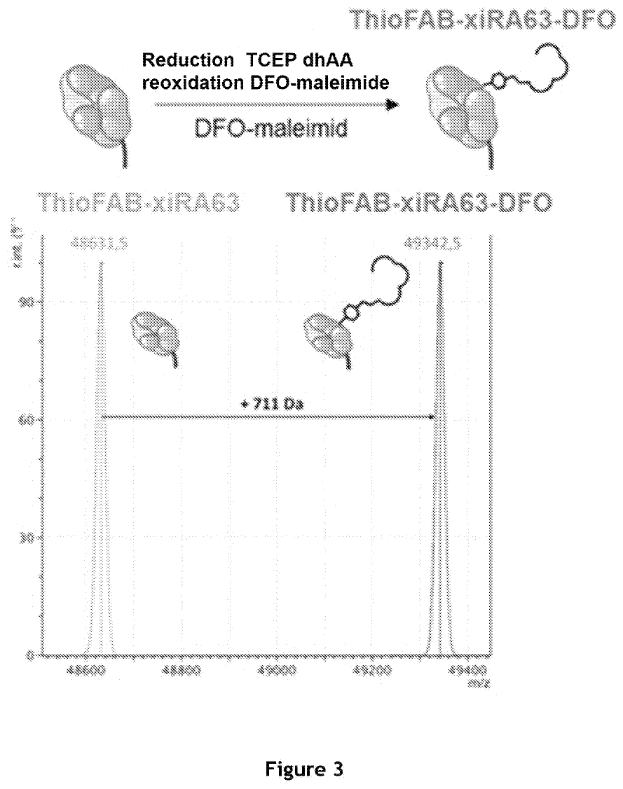

FIG. 2 shows antigen binding curves comparing the mutant Fab fragment with a cysteine at position 128 (IMGT) of the heavy chain, according to the IMGT nomenclature, of the xiRA63 antibody and a conjugate obtained by site-specific grafting of a bifunctionalised tetrazine with a Zirconium-89 chelating agent (DFO) and a fluorophore (IRDye800).

In addition, Tables 6 and 7 below show the values of Bmax and Kd obtained from the curves.

TABLE 6

| | Non-mutated Fab | Random conjugate |
|---|---|---|
| Bmax | 70.39 | 28.43 |
| Kd | 1.811 | 2.9712 |

TABLE 7

| | Mutant Fab | Site-specific conjugate |
|---|---|---|
| Bmax | 63.64 | 55.27 |
| Kd | 0.9418 | 0.4562 |

Whereas random conjugation of the unmutated Fab results in a loss of target affinity and recognition (see FIG. 1 and Table 6), site-specific conjugation of the mutant Fab with a cysteine at position 128 of the heavy chain according to IMGT nomenclature does not induce a significant change in antigen binding compared with the native mutant Fab (see FIG. 2 and Table 7).

### Conclusion

The site-specific conjugate obtained from the mutant Fab with a cysteine in position 128 of the heavy chain according to the IMGT nomenclature has better conjugation characteristics than the random conjugate obtained from the conventional Fab, which is extremely disrupted. Because of its much better properties, the clinical use of the site-specific conjugate obtained from the Fab mutant with a cysteine in position 128 of the heavy chain according to the IMGT nomenclature may then be possible.

### Example 3: Preparation of a Fab Mutant of the Chimeric Antibody xiRA63 with a Cysteine in Position 128 (IMGT Nomenclature) of the Heavy Chain, Grafting, In Vitro Characterisation and In Vivo Use for Imaging in a Glioblastomamodel

### Materials and Methods

### Production of ThioFab-xiRA63:

The heavy and light chains of ThioFab-xiRA63 were cloned into the eukaryotic expression vector pTT5 (Durocher et al., 2002). Plasmids were co-transfected into ExpiCHO-S cells (ThermoFisher) according to the manufacturer's “ExpiCHO™ Expression System User Guide” (MAN0014337 ThermoFisher Scientific). Day 12 post-transfection, the supernatant was clarified by centrifugation at 4000-5000×g for 30 min at 4° C. and filtered through a 0.22 pm filter. ThioFab xiRA63 was then affinity purified on a HiTrap KappaSelect column (GE Healthcare). After elution, antibody solutions were dialysed with the Slide-A-Lyzer™ G2 dialysis cassette (Thermoscientific) with a cut-off of 10 kDa in 1 litre of PBS.

### Conjugation of ThioFab-xiRA63-DFO:

ThioFab-xiRA63 (500 μg, 459 μL at 1.1 g/L) was reduced with 30.8 μL TCEP (20 mM) at 37° C. for 1 hour. After purification using centrifugal filter units with a molecular weight cut-off of 10,000 Da, reoxidation of the native interchain disulfide bond was carried out with 6.75 μL dehydroascorbic acid (dhAA, 20 equiv., 50 mmol/L in carbonate/bicarbonate buffer pH 9) for 1 hour at 37° C. After removing excess dhAA with centrifugal filtration units, 3.62 μL of deferoxamine-maleimide (10 equiv., 15 mmol/L in DMSO) was added to the reaction mixture and stirred at 37° C. for 2 hours. The conjugate was then purified by steric exclusion chromatography (Superdex 75 10/300 GL) to remove potential dimers. Validation of DFO conjugation on ThioFab-xiRA63 was carried out by mass spectrometry (MALDI-TOF).

Radiolabelling of ThioFab-xiRA63-DFO with Zirconium 89 [⁸⁹Zr]:

In order to radiolabel this antibody fragment with zirconium-89 (PerkinElmer), a protocol previously described by Vosjan et al., 2010 (Vosjan, M., Perk, L., Visser, G. et al. Conjugation and radiolabeling of monoclonal antibodies with zirconium-89 for PET imaging using the bifunctional chelate p-isothiocyanatobenzyl-desferrioxarnine. Nat Protoc 5, 739-743 (2010). https://doi.org/10.1038/nprot.2010.13) was used. The day before the injection of 0.65 rng ThioFab-xiRA63-DFO, pH adjusted to 7.2, was incubated with the radioelement ⁸⁹Zr for 1 hour at 37° C. with stirring at 300 rpm. The oxalic acid in the ⁸⁹Zr solution (274 μL, 11.17 mCi) was neutralised with 90 μL Na₂CO₃ (2 M) before adding to the antibody solution. The [⁸⁹Zr]Zr-ThioFab-xiRA63-DFO was then purified on a PD-10 column with gentisic acid solution (5 mg/mL in 0.25 M sodium acetate, pH 5.5) as the mobile phase and concentrated with Vivaspino ultrafiltration tubes (Satorius) with a 10 kDa cut-off.

### Evaluation of the Radiochemical Purity of the [⁸⁹Zr]Zr-ThioFab-xiRA63-DFO Fragment b HPLC:

The radiochemical purity of the radioligand was verified via size-exclusion high-performance liquid chromatography (HPLC), coupled with a UV (UVD 170U UV/VIS) and gamma scintillation detector (Packard). Measurements were carried out on a bioZen 1.8 μm SEC-2 LC analytical column (Phenomenex) coupled to a DIONEX HPLC system (Thermo-Fisher). Elution was carried out using a linear gradient with a solution of KH₂PO₄ (50 mM) and KCl (250 mM) (pH 6.8), at a flow rate of 0.2 mL/min.

Analysis of the [⁸⁹Zr]Zr-ThioFab-xiRA63-DFO fragment by flow cytometry:

We used the protocol of Herbet et al. 2018 (Herbet A. et al. Antibodies Targeting Human Endothelin-1 Receptors Reveal Different Conformational States in Cancer Cells. Physiol. Res. 67 (Suppl. 1): 5257-5264, 2018. https://doi.org/10.33549/physiolres.933848). Two cell lines were used to assess the apparent affinity of the different antibody formats: Chinese Hamster Ovary Wild-Type (CHO-WT) and Chinese Hamster Ovary stably transfected with human ETA (CHO-ETA). Cells were resuspended in saturation buffer (1×PBS, 5% normal goat serum, 0.1% bovine serum albumin) and 3.10¹ live cells were seeded in each tube. Apparent affinity was determined with a dilution range of 0.01 nM to 50 nM for the ThioFab-xiRA63-DFO and ThioFab-xiRA63 fragments. The samples were incubated at 4° C. overnight. The following day, the system was revealed by a secondary goat anti-human IgG antibody Fd (CliniSciences 2046-02) conjugated to a fluorescent molecule, FITC, diluted 500^{th} and incubated for 4 hours at 4° C. The samples were washed three times with PBS between each step. Sample fluorescence was measured at a wavelength of 488 nm using the FACSCalibur flow cytometer (BD Biosciences, San Jose, CA, USA) and expressed as a percentage of the mean fluorescence intensity per sample. The “one site specific binding” function in GraphPad was applied to obtain the binding curve, Bmax and apparent Kd±SEM.

### Preclinical Model:

All animal experiments were carried out in accordance with European Directive 2010/63/EU and its transposition into French law (Decree no. 2013-118). Six four-week-old female nude, NMRI mice (Janvier Labs) were orthotopically implanted with 5.10⁵ glioblastoma cells (Gli7; ET_{A}⁺), in the striatum (2 mm to the right of the bregma and at 2.5 mm depth of the dura) under anaesthesia with isoflurane combined with xylocaine locally. A 10 μL Hamilton syringe was used with a flow rate of 0.25 μL/min. During injection, 0.05 mg·kg⁻¹ buprenorphine was administered subcutaneously to each mouse. The mice were housed in groups of four in ventilated cages with a humidity of 40% and a temperature-controlled room at 22° C.

### Antibody Injection:

78 days after glioblastoma cell implantation (Herbet A. et al. Antibodies Targeting Human Endothelin-1 Receptors Reveal Different Conformational States in Cancer Cells. Physiol. Res. 67 (Suppl. 1): 5257-5264, 2018. https://doi.org/10.33549/physiolres.933848), a total of 5 mice received an intravenous bolus of [⁸⁹Zr]Zr-ThioFab-xiRA63 (100 μC±5 μC).

### PET-CT Image Acquisition:

An initial 60-minute dynamic acquisition was performed immediately after antibody injection, using an Inveon microPET scanner or an Inveon microPET/CT scanner (Siemens). A 20-minute PET scan was then performed at 5 hours, 24 hours, 48 hours, 72 hours and 7 days post-injection. The mice were imaged under anaesthetic.

### PET-CT Analysis:

The PMOD software (Version 3.9) was used to analyse the various acquisitions. Spherical volumes of interest (Vis) with a fixed size of 8 mm³ were created in a plurality of organs: heart, liver, kidney, spleen and muscle. For bone and brain tumours, the Vis were plotted using the “iso-contour” tool. The same VI as applied to the tumour side were applied to the side contralateral to the tumour (used as a negative control). The mean volume activity (kBq·cm⁻³) in each VI was corrected for the half-life of ⁸⁹Zr (t_{1/2}=3.3 days) following the radioactive decay law. Then, to quantify the antibody, said mean volume activity was divided by the total activity injected to obtain the percentage of dose injected per volume of tissue (% DI·cm⁻³).

### Statistics:

The GraphPad Prism (v9.0.1) software was used for the statistical analysis. Student's t-test (two-tailed) was performed to compare two groups of data (*P<0.05, **P<0.01, ***P<0.001, ****P<0.0001).

### Results

### Validation of DFO Antibodies by Mass Spectrometry

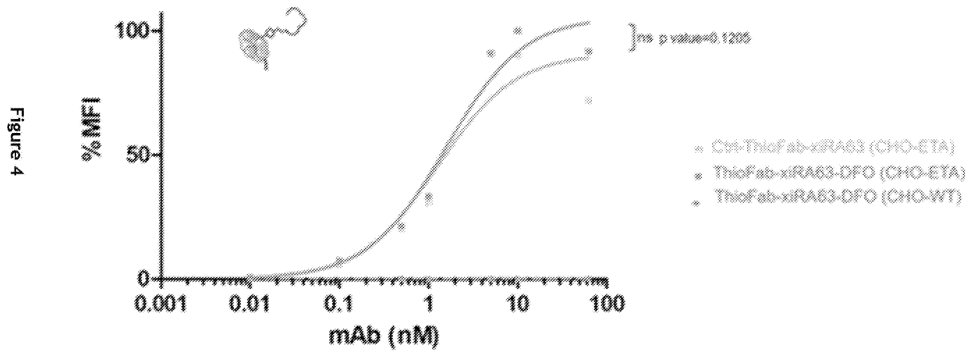

ThioFab-xiRA63 has an equivalent m/z of 48631.5 daltons while ThioFab-xiRA63-DFO has an m/z of 49342.5 daltons. The difference of 711 daltons corresponds perfectly to the molecular weight of DFO-maleimide (FIG. 3).

### Validation of DFO Antibodies by Flow Cytometry:

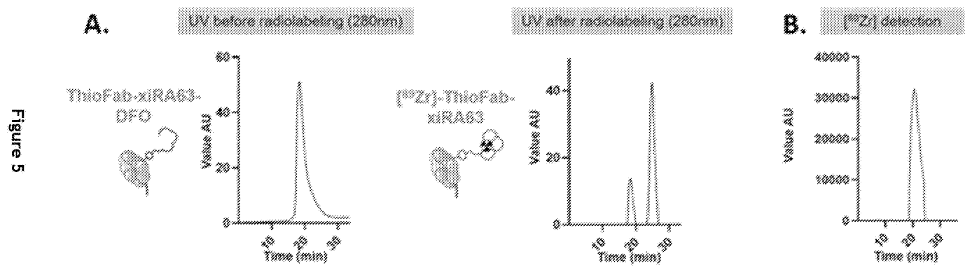

ThioFab-xiRA63 with and without DFO show high affinity for human ET_{A} receptors (see FIG. 4), with similar apparent affinity constants in the nanomolar range (Kd) showing that DFO conjugation on the additional free thiol does not alter the affinity of this fragment for its target (p-value>0.1). Their specificity was tested with CHO-WT negative control cells and CHO-ET_{A}positive control cells. The signal obtained on CHO-WT is in the background noise, i.e. less than 2% of the MFI obtained with the ThioFab xiRA63-DFO concentration.

### PET Imaging

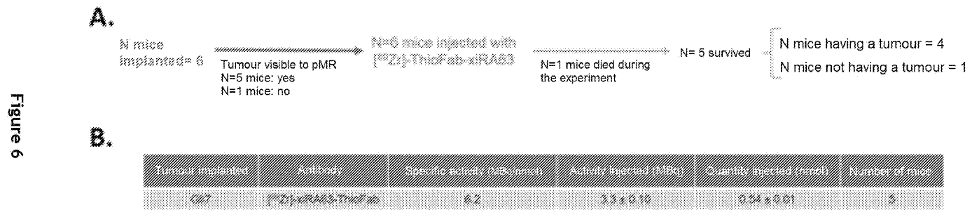

For the PET imaging experiments, ThioFab-xiRA63-DFO was radiolabelled with Zirconium 89 [⁸⁹Zr] according to the procedure previously published by Vosjan et al. (Vosjan, M., Perk, L., Visser, G. et al. Conjugation and radiolabeling of monoclonal antibodies with zirconium-89 for PET imaging using the bifunctional chelate p-isothiocyanatobenzyl-desferrioxamine. Nat Protoc 5, 739-743 (2010). https://doi.org/10.1038/nprot.2010.13). Their radiochemical purity was assessed by size-exclusion high-performance liquid chromatography (HPLC), and was estimated to be 100% (FIG. 5).

### Preclinical Model

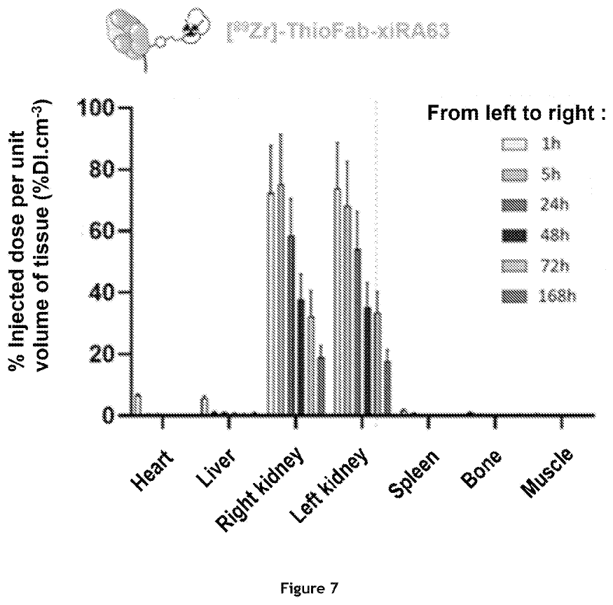

Once the radiolabelled antibody ([⁸⁹Zr]Zr-ThioFab-xiRA63) was validated, it was injected (5.1±0.13 MBq, 3.3±0.1 nmol) into mice (n=5) that had been previously orthotopically xenografted with glioblastoma tumour cells, named Gli7, expressing ET_{A} at their membrane (ET_{A}+, Herbet A. et al. Antibodies Targeting Human Endothelin-1 Receptors Reveal Different Conformational States in Cancer Cells. Physiol. Res. 67 (Suppl. 1): 5257-5264, 2018. https://doi.org/10.33549/physiolres.933848) (FIG. 6). Whole body images of the mice were then captured by microPET/CT at various times (1 hour after injection, then at 5 hours, 24 hours, 48 hours, 72 hours, and 168 hours, i.e. 7 days p.i.), enabling the study of the tissue distribution (biodistribution) of our PET immunotracer over time.

In this experiment (FIG. 7), [⁸⁹Zr]Zr-ThioFab-xiRA63 (<60 kDa) appears to follow a renal elimination pathway (70% ID·cm⁻³ accumulation in the kidney in the first hour p.i.). Small molecules were rapidly eliminated from the bloodstream by glomerular filtration and renal excretion. Our tracer follows one of the classic elimination routes due to its low molecular weight, without targeting non-specific organs in which the murine endothelin A receptor might be found (muscle, bone, bladder, ovaries, etc.).

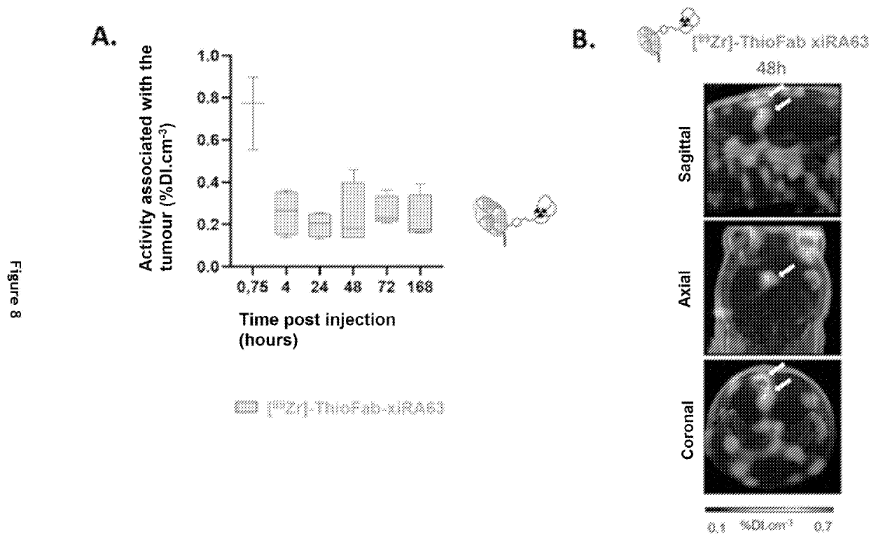

Detection of the accumulation of [⁸⁹Zr]Zr-ThioFab-xiRA63 in Gli 7 brain tumours (FIG. 8A), can be seen as early as 1 hour pi. (−1% DI·cm⁻³). Due to its rapid removal from plasma and blood, its signal weakens rapidly and is close to −0.3% DI·cm⁻³ after 5 hours pi.. However, this rapid removal enables better contrast in order to visualise the tumour due to the low spurious signals and the virtual absence of background noise (FIG. 8B).

### Conclusion

We have demonstrated the functionality of the conjugated fragment [Zr⁸⁹]Zr-ThioFab-xiRA63-DFO in vivo. It is capable of recognising brain tumours expressing endothelin receptors. Its biodistribution is also consistent with a Fab fragment (mutant or non-mutant) and with molecules with a molecular weight of less than 60 kDa, i.e. rapid removal by the kidneys.

### Example 4: Preparation of a thiofab (thioFab-xiRB49) of the Chimerised RB49 Antibody (xiRB49)

Unexpectedly, simple chimerisation (substitution of mouse constant regions with human constant regions) of RB49 led to a non-functional chimeric antibody, unable to bind specifically to the ET_{B} receptor. Studies in silico have shown that the presence of an unusual proline at position 125 of the variable region of the RB49 heavy chain could induce a different folding of the variable region during its chimerisation. The heavy and light chain CDR3s then come into prolonged contact, making them unavailable for interaction with the ET_{B} antigen. Mutation of P125 by a canonical threonine restored the functionality of the chimeric antibody (xiRB49) (Marie Hautiere et al., (2023) The functionality of a therapeutic antibody candidate restored by a single mutation from proline to threonine in the variable domain, Human Vaccines & Imnmunotherapeutics, 19:3).

These results suggest that the affinity of the RB49 antibody for its ET_{B} antigen may be affected by a single mutation in the VH domain (FR4 portion). They highlight the structural complexity of the RB49 antibody, making it difficult to predict the effect of any modification.

Despite a change in the conformation of the Kappa constant region associated with the substitution of the alanine at IMGT position 128 by a cysteine (A128C IMGT), it is demonstrated here that a Fab of this antibody also comprising a mutation of the cysteine at IMGT position 128 of the VH domain (thioFab-xiRB49) retains its affinity for its ET_{B} antigen.

### Materials and Methods

Production and Purification of the Chimeric Antibodies xiRB49-P125T, Fab-xiRB49-P125T and ThioFab-xiRB49-P125T

A chimeric version of RB49 called xiRB49 was generated. The heavy and light chains encoding IgG-xiRB49, Fab-xiRB49-P125T and ThioFab-xiRB49-P125T were subcloned into the pTT5 expression plasmid. The vectors were co-transfected into ExpiCHO-S cells (ThermoFisher Scientific) using the ExpiCHO Expression System kit (ThermoFisher Scientific) according to the manufacturer's instructions (MAN0014337 ThermoFisher Scientific). At day 12 post-transfection, the supernatant was clarified by centrifugation at 4000-5000×g for 30 min at 4° C. and filtered through a 0.22 μm filter. xiRA63 was purified on a HiTrap Protein A HP column (GE HealthCare) and Fab and ThioFab on HiTrap KappaSelect (GE HealthCare). After elution, antibody solutions were dialysed with the Slide-A-Lyzer™ G2 dialysis cassette (ThermoFisher Scientific) in 1 L of phosphate-buffered saline (PBS). Thermal stability study

Samples at 1 mg-mL-1 were deposited in capillaries to determine antibody denaturation temperature curves (Tycho NT6 instrument, NanoTemper). The A350 nm/A330 nm ratio was calculated, and its first derivative gave us an inflection temperature (Ti) corresponding to the change in conformation of the protein domain from the native state to the denatured state. Determination of antibody binding curves on ET_{B} with a goat polyclonal anti-Human Kappa secondary antibody.

The binding experiments were performed on CHO cells expressing or not expressing endothelin receptors. Cells were incubated overnight at 4° C. with Fab-xiRB49-P125T or ThioFab-xiRB49-P125T ranging from 0.01 nM to 150 nM and detected with an Alexa Fluor 488-conjugated polyclonal goat anti-human Kappa secondary antibody (A18854). Throughout this experiment, three washes with 1×PBS (pH: 7.4) were performed between each step to remove the non-specific signal.

### Competition Test

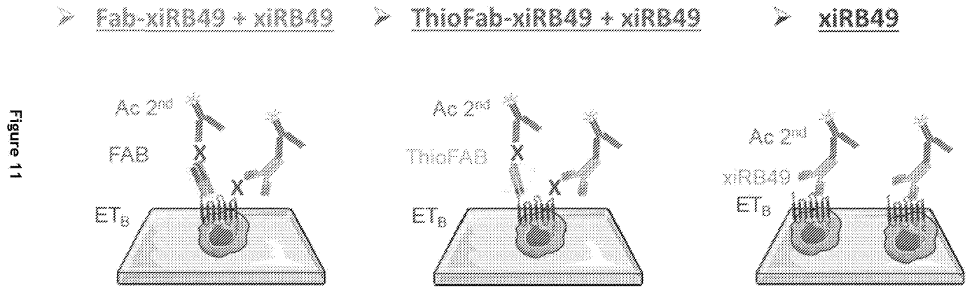

Cells were pre-incubated overnight at 4° C. in the presence of a molar excess (500 nM) of Fab-xiRB49-P125T or ThioFab-xiRB49-P125T antibody fragments. Then, a control range of xiRB49-P125T antibody was added for each of the conditions (see FIG. 11). Ranges read by flow cytometry were revealed by Alexa Fluor488-coupled goat polyclonal anti-human IgG secondary antibody (1110120) targeting only the Fc regions of the antibodies. Throughout this experiment, three washes with 1×PBS (pH: 7.4) were performed between each step to remove the non-specific signal.

Determination of Antibody Binding Curves on ET_{B} with an Anti-Fd Secondary Antibody

The binding experiments were performed on CHO cells expressing or not expressing endothelin receptors. Cells were incubated overnight at 4° C. with Fab-xiRB49-P125T or ThioFab-xiRB49-P125T ranging from 0.01 nM to 150 nM and detected with an FITC-conjugated goat polyclonal anti-human Fd-IgG secondary antibody (Southern Biotech™ 204602). Throughout this experiment, three washes with 1×PBS (pH: 7.4) were performed between each step to remove the non-specific signal.

### Results

### Physico-Chemical Parameters of ThioFab-xiRB49-P125T

Once the vectors needed for its production and purification had been constructed, the physico-chemical properties of ThioFab-xiRB49-P125T were determined. The ThioFab-xiRB49-P125T fragment, like the ThioFab-xiRA63 fragment, had an expected molecular weight of 50 kDa with a very acceptable purity (>95%) assessed by SDS PAGE. The hydrodynamic radius obtained by DLS was approximately 2.6 nm±0.1 nm and identical to that of all the Fabs obtained (results not shown).

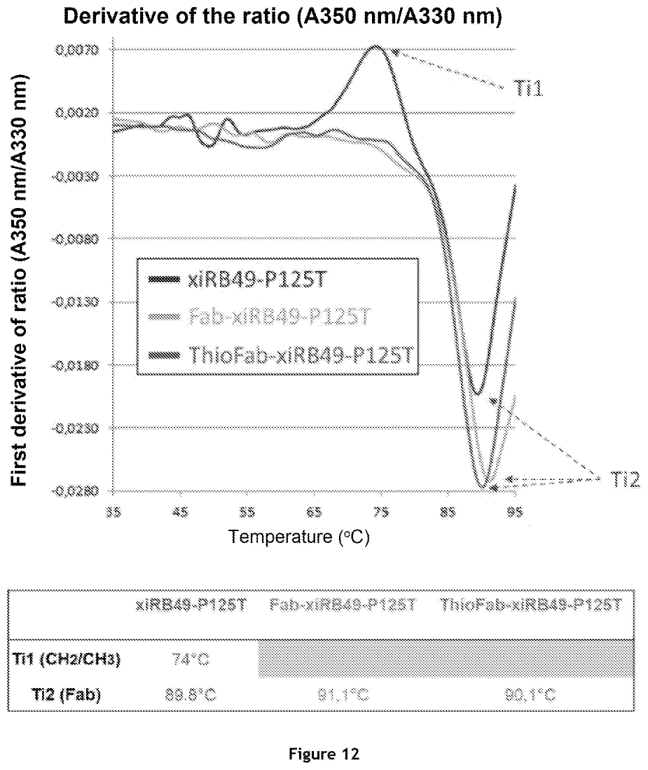

The thermal stability of the antibodies was studied and thermal denaturation curves were obtained (Tycho NT, Nanotemper). FIG. 12 shows the first derivative of the A350 nm/A330 nm ratio of xiRB49-P125T, Fab-xiRB49-P125T and ThioFab-xiRB49-P125T antibodies, enabling the inflection temperatures (T₁) to be obtained. All three antibodies show the same T₁2, reflecting the Fab domain of around 90° C. After confirming the physico-chemical properties of the different fragments, their functionality was assessed.

### Antibody Functional Studies: First Determination of Antibody Binding Curves on ET_{B}.

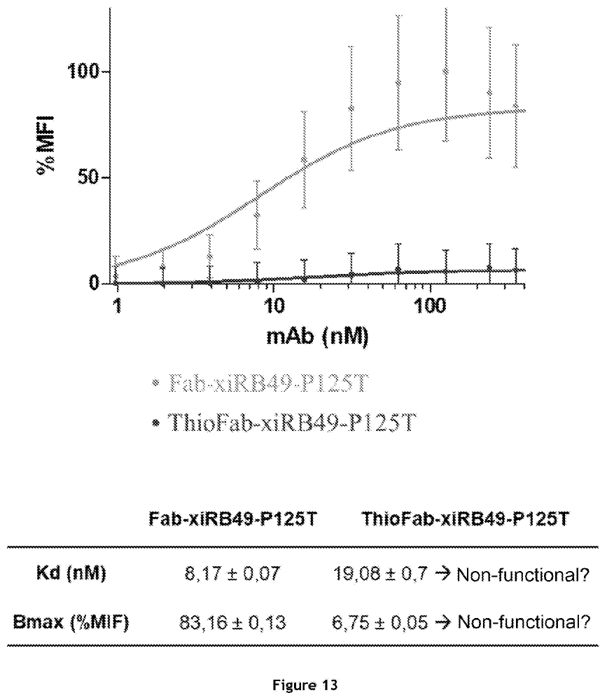

The functionality of Fab-xiRB49-P125T and ThioFab-xiRB49-P125T was assessed by producing binding curves by flow cytometry on CHO-ETB cells (FIG. 13). The results show that Fab-xiRB49-P125T has a high apparent affinity in the nanomolar range and a Bmax close to 83% MFI (FIG. 13).

Surprisingly, no binding curve was obtained for ThioFab-xiRB49-P125T (FIG. 13) whereas its T₁2 indicates that its Fab domain appears to be folded in the same manner as the corresponding antibody and Fab, suggesting that the ThioFab should be functional. It was hypothesised that ThioFab-xiRB49-P125T was not recognised by the secondary antibody. In this binding experiment, the Fabs are revealed by a goat polyclonal anti-human-Kappa-AlexaFluor 488 secondary antibody (ref A18854). It is possible that the cysteine added at the end of the variable region, IMGT position A128C, could have modified this interaction which is necessary for its detection.

### Functional Validation of ThioFab-xiRB49-P125T

In order to test this hypothesis, a competition test between xiRB49-P125T and Fab-xiRB49-P125T or ThioFab-xiRB49-P125T was developed (cf. Appendix—Supplementary material and method section). CHO-ETB cells were pre-incubated overnight in the presence of Fab-xiRB49-P125T or ThioFab-xiRB49-P125T fragments at a concentration of 500 nM. The next day, after washing, xiRB49-P125T was added in increasing concentrations. Thus, three conditions were tested (FIG. 11):
1. A reference condition (positive control) with CHO-ETB cells pre-incubated in the presence of PBS (vehicle) and therefore without signal shift on addition of xiRB49-P125T at different concentrations;
2. A control condition for the validity of the competition test: as the Fab-xiRB49-P125T fragment is functional, it should interfere with the attachment of xiRB49-P125T to ET_{B}. Thus, a higher concentration of xiRB49-P125T antibody will be required to displace this signal, resulting in a change in apparent Kd.
3. A test condition with the ThioFab-xiRB49-P125T fragment. If the effect observed with the Fab-xiRB49-P125T condition is similar to that obtained with ThioFab-xiRB49-P125T, then this antibody is functional but not recognised by the anti-human-Kappa secondary antibody.

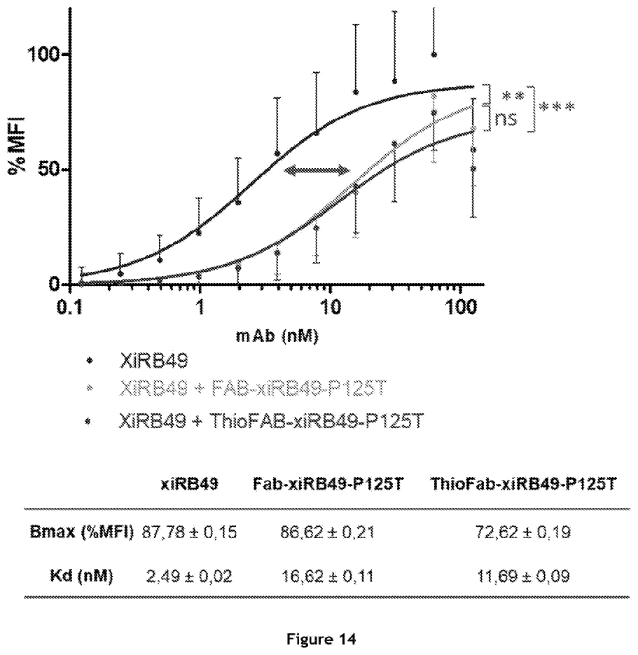

FIG. 14 shows the results of the binding curves obtained with the competition assay. As expected, Fab-xiRB49-P125T displaced the signal emitted by xiRB49-P125T, with apparent affinity increasing from 2.49 nM to 16.62 nM. Similarly, ThioFab-xiRB49-P125T induced an equivalent shift in the signal emitted by xiRB49-P125T, with the apparent Kd rising to 11.69 nM. No significant difference was observed between the two competition conditions involving Fab-xiRB49-P125T and ThioFab-xiRB49-P125T.

This result proves that the ThioFAb-xiRB49-P125T antibody is functional as suggested by its thermal denaturation curve and its inflection temperature Tᵢ2.

Indirectly, the absence of recognition of ThioFab-xiRB49-P125T by an anti-human-kappa-polyclonal secondary antibody strongly suggests that the addition of the cysteine nevertheless modifies the conformation of the kappa constant region.

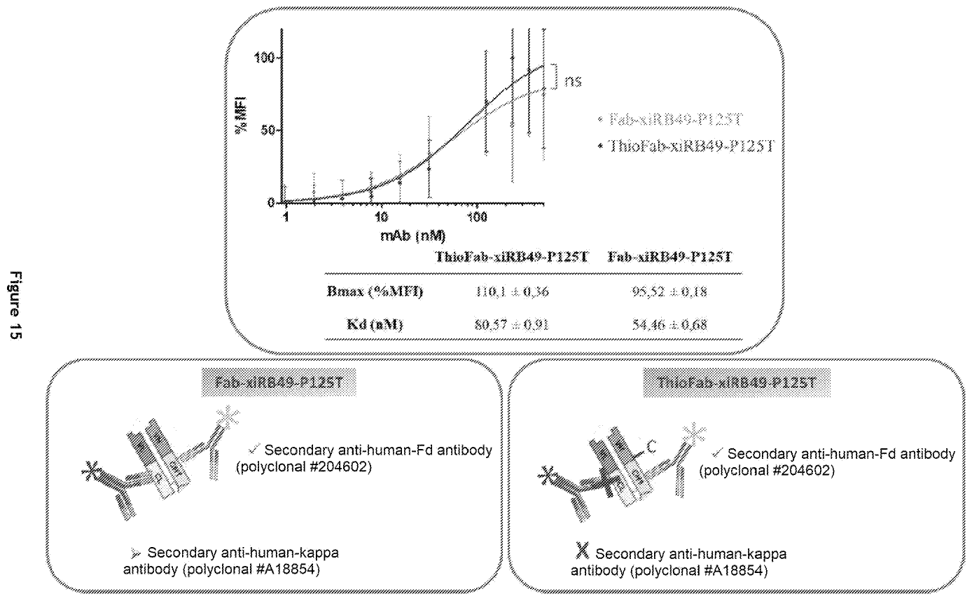

A new secondary antibody, this time targeting the Fd region of the antibodies (CH1+VH), was used to validate the functionality of ThioFab-xiRB49-P125T. Under these conditions, binding to the ET_{B} antigen of ThioFAb-xiRB49-P125T is well demonstrated (FIG. 15), with an apparent Kd and Bmax similar to Fab-xiRB49-P125T.

Taken together, these results support the hypothesis that the cysteine at IMGT position 128 modifies the Kappa region in ThioFab-xiRB49-P125T, which is then no longer recognised by a secondary anti-human-kappa-polyclonal antibody. However, ThioFab-xiRB49-P125T retains an intact ability to bind the ET_{B} antigen.

### Conclusion

Despite the effect of abolishing the affinity for the ET_{B} antigen bonded by a single chimerisation, substitution of the proline at IMGT position 125 by a threonine (IMGT P125T) was able to restore the affinity for the ET_{B} antigen of the chimeric antibody (xiRB49-P125T) and of a Fab fragment of such a chimeric antibody (Fab-xiRB49-P125T).

Furthermore, despite a partial destructuring of the kappa region (which is then no longer recognised by a polyclonal anti-human-kappa secondary antibody) associated with the additional substitution in Fab-xiRB49-P125T of the alanine at IMGT position 128 with a cysteine (IMGT A128C), this substitution does not affect the affinity for the ET_{B} antigen of ThioFab-xiRB49-P125T. Strangely, this destructuring of the kappa region was not found in ThioFab-xiRA63 following the insertion of C121, suggesting a phenomenon specific to the variable regions of RB49.

1. A mutant antibody fragment specifically binding to the endothelin A receptor or endothelin B receptor, comprising a mutated heavy chain variable domain, the amino acid residue at position 128 according to the IMGT nomenclature of the heavy chain variable domain being substituted by a cysteine, provided that said fragment is not a Fab fragment in which the mutated heavy chain fragment corresponds to the amino acid sequence SEQ ID NO: 1 and the light chain fragment corresponds to the amino acid sequence SEQ ID NO: 2.

2. The mutant antibody fragment according to claim 1, which further comprises a light chain variable domain.

3. The mutant antibody fragment according to claim 2, which is a Fab fragment.

4. The mutant antibody fragment according to claim 1, which does not comprise a light chain variable domain.

5. The mutant antibody fragment according to claim 1, which is chimeric, humanised or human.

6. The mutant antibody fragment according to claim 1, wherein the antibody fragment binds specifically to the endothelin B receptor, and comprises:
a heavy chain variable domain (VH) comprising three heavy chain CDRs, namely CDR1-H, CDR2-H and CDR3-H having according to the IMGT nomenclature for respective sequences GYTFISYW (SEQ ID NO: 3), IDPDSGGT (SEQ ID NO: 4) and AREGDYAWFAY (SEQ ID NO: 5), or a sequence with at least 80% identity with GYTFISYW (SEQ ID NO: 3), IDPDSGGT (SEQ ID NO: 4) or AREGDYAWFAY (SEQ ID NO: 5), and
a light chain (VL) variable domain comprising three light chain CDRs, namely CDR1-L, CDR2-L and CDR3-L having according to the IMGT nomenclature for respective sequences QSIVHSNGNTY (SEQ ID NO: 6), KVS (SEQ ID NO: 7) and FQGSHVPWT (SEQ ID NO: 8) or a sequence with at least 80% identity with QSIVHSNGNTY (SEQ ID NO: 6), KVS (SEQ ID NO: 7) or FQGSHVPWT (SEQ ID NO: 8).

7. A mutant antibody fragment according to claim 1, wherein the antibody fragment binds specifically to the endothelin A receptor, and comprises:
a VH domain comprising three heavy chain CDRs, namely CDR1-H, CDR2-H and CDR3-H having according to the IMGT nomenclature for respective sequences GFTFNIYA (SEQ ID NO: 9), IRSKSNNYAT (SEQ ID NO: 10) and VSSYYSGSFFAY (SEQ ID NO: 11), or a sequence with at least 80% identity with GFTFNIYA (SEQ ID NO: 9), IRSKSNNYAT (SEQ ID NO: 10) or VSSYYSGSFFAY (SEQ ID NO: 11), and
a VL domain comprising three light chain CDRs, namely CDR1-L, CDR2-L and CDR3-L having, according to the IMGT nomenclature for respective sequences SQSIVYSNGKIYL (SEQ ID NO: 12), KVS (SEQ ID NO: 13) and FQGSHLPLT (SEQ ID NO: 14) or a sequence with at least 80% identity with SQSIVYSNGKIYL (SEQ ID NO: 12), KVS (SEQ ID NO: 13) or FQGSHLPLT (SEQ ID NO: 14).

8. The mutant antibody fragment according to claim 1, wherein the antibody fragment binds specifically to the endothelin B receptor, and comprises:
a VH domain possessing the sequence SEQ ID NO: 15 or a sequence having at least 80% identity with SEQ ID NO: 15, and
a VL domain possessing the sequence SEQ ID NO: 16 or a sequence having at least 80% identity with SEQ ID NO: 16.

9. A mutant antibody fragment according to claim 7, wherein the antibody fragment binds specifically to the endothelin A receptor, and comprises:
a VH domain possessing the sequence SEQ ID NO: 17 or a sequence having at least 80% identity with SEQ ID NO: 17, and
a VL domain possessing the sequence SEQ ID NO: 18 or a sequence having at least 80% identity with SEQ ID NO: 18.

10. A nucleic acid molecule or pair of nucleic acid molecules encoding the mutant antibody fragment of claim 1.

11. A vector comprising the nucleic acid molecule or pair of nucleic acid molecules of claim 10.

12. A host cell, a transgenic non-human animal or a transgenic plant comprising the nucleic acid molecule or pair of nucleic acid molecules of claim 10.

13. A conjugate comprising the mutant antibody fragment according to claim 1, bonded to at least one molecules of interest, via the cysteine in position 128 of the heavy chain variable domain according to the IMGT nomenclature of the mutant antibody fragment.

14. The conjugate according to claim 13, corresponding to the following formula (I), (IIa) or (IIb) 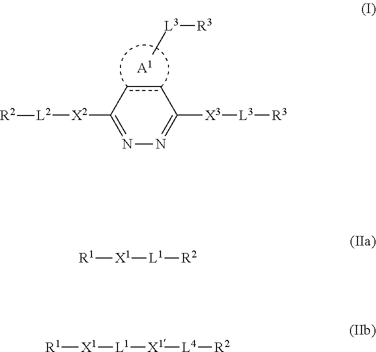
in which:
R¹ represents a mutant antibody fragment according to claim 1 bonded to X¹ via the cysteine, and more particularly the thiol function of the cysteine, in position 128 of the heavy chain variable domain according to the IMGT nomenclature;
X¹ represents 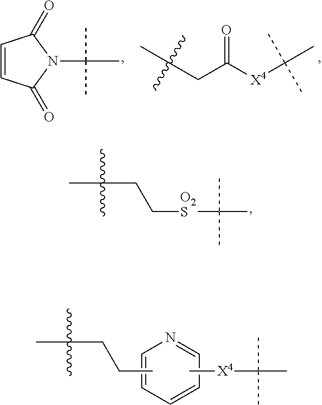
with X⁴ representing a single bond, O, S, or NR⁴, and R⁴ representing H or (C₁-C₆)alkyl, the wavy bond indicating the point of attachment to R¹ and the dashed bond indicating the point of attachment to L¹;
X^{1′} represents 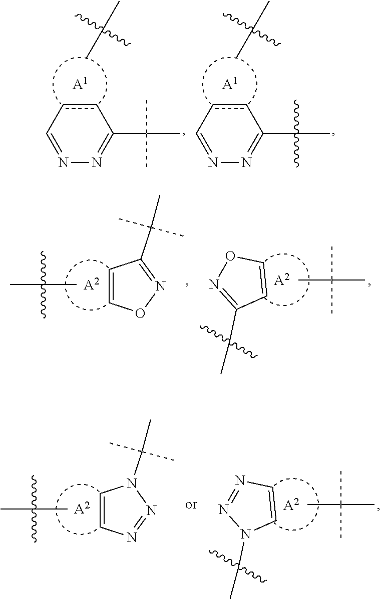
with the wavy bond indicating the point of attachment to L⁴, and the dashed bond indicating the point of attachment to L¹;
L¹, L², L³ and L⁴ each independently represent a single bond or a spacer, the spacer being a (C₁-C₃₀)-alkyl chain, optionally preceded and/or interrupted and/or followed by one or more units selected from the group consisting of aromatic rings, heteroaromatic rings, cycloalkane rings, cycloalkene rings, heterocycloalkane rings, heterocycloalkene rings, —O—, —S—, —NR⁵—, —C(O)—, —C(S)—, —C≡C—, and —C(R⁶)═C(R⁷)—, with R⁵ being H or (C₁-C₆)alkyl, where R⁶ and R⁷ are each independently H or (C₁-C₆)alkyl, and the aromatic, heteroaromatic, cycloalkyl, cycloalkenyl, heterocycloalkyl and heterocycloalkenyl rings being optionally substituted by one or more (C₁-C₆)alkyl groups; [symbol]
represents a double bond or a single bond;
ring A¹ represents a cycloalkane, cycloalkene, heterocycloalkane or heterocycloalkene ring, optionally fused with one or more aromatic rings and optionally substituted with one or more groups chosen from (C₁-C₆)alkyl, halide and hydroxy;
ring A² represents a cycloalkene or heterocycloalkene ring, optionally fused with one or more aromatic rings and optionally substituted with one or more groups chosen from (C₁-C₆)alkyl, halide and hydroxy;
X² and X³, identical or different, each represent 0, S or NR⁸, with R⁸ representing H or (C₁-C₆)alkyl;
R² and R³, identical or different each represent a residue of a molecule of interest.

15. The conjugate of claim 14, wherein the molecule of interest is:
a molecule that is detectable or able to become detectable, chosen from a fluorophore, a chromophore, an affinity molecule, a radioisotope and a chelating agent;
an affinity molecule chosen from biotin, avidin, streptavidin and a hexahistidine peptide; and/or
a pharmacomodulatory molecule chosen from compounds bearing linear or branched chains of poly(ethylene glycol), linear or branched chains of poly(glutamic acid), cholesterol, and molecules capable of binding to albumin.

16. The conjugate according to claim 14, having formula (I) and wherein at least one of R¹ and R² is a residue of a molecule which is detectable or able to become detectable.

17. A method for preparing a conjugate according to claim 13 comprising the following steps:
a) reducing the disulfide bridge or bridges of the mutant antibody fragment to give a reduced mutant antibody fragment,
b) re-oxidising the disulfide bridge or bridges of the mutant antibody fragment to give a partially reduced mutant antibody fragment,
c) conjugating, with at least one molecule of interest, the thiol function of the cysteine in position 128 of the heavy chain variable domain according to the IMGT nomenclature of the partially reduced mutant antibody fragment, to give a conjugate according to claim 13.

18. The method according to claim 17, wherein:
the conjugate is a conjugate of formula (I) according to claim 14 and step c) is carried out by coupling between the partially reduced mutant antibody fragment and a compound of the following formula (III): 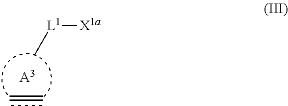
wherein L¹ is as defined in claim 14, ring A³ is a cycloalkyne, cycloalkene, heterocycloalkyne or heterocycloalkene ring, optionally fused with one or more aromatic rings and optionally substituted with one or more groups from (C₁-C₆)alkyl, halide, and hydroxy, and X^{1a} is a chemical group reactive towards an SH function, to give a compound of the following formula (IV): 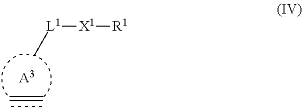
wherein R¹, X¹ and L¹ are as defined in claim 14, and ring A³ is as defined above, followed by coupling between the compound of formula (IV) and a compound of the following formula (V): 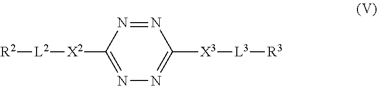
wherein R², R³, L², L³, X² and X³ are as defined in claim 14; or
the conjugate is a conjugate of formula (IIa) according to claim 14 and step c) is carried out by coupling the partially reduced mutant antibody fragment with a compound of formula X^{1a}-L¹-R² wherein L¹ and R² are as defined in claim 14 and X^{1a} is as defined above; or
the conjugate is a conjugate of formula (IIb) according to claim 14 and step c) is carried out:
i) by coupling the partially reduced mutant antibody fragment with a compound of formula X^{1a}-L¹-X^{1′}-L⁴-R² wherein L¹, L⁴, X^{1′} and R² are as defined in claim 14 and X^{1a} is as defined above; or
ii) by coupling the partially reduced mutant antibody fragment with a compound of formula X^{1a}-L¹-G¹ wherein X^{1a} is as defined above, L¹ is as defined in claim 14 and G¹ represents a reactive chemical group, to give a conjugate of formula R¹—X¹-L¹-G¹, followed by coupling between the conjugate of formula R¹—X¹-L¹-G¹ and a compound of formula G²-L⁴-R² wherein L⁴ and R² are as defined in claim 14 and G² is a reactive chemical group different from G¹ and capable of reacting with G¹ to form a group X^{1′}.

19. An intermediate conjugate comprising the mutant antibody fragment according to claim 1, bonded to a reactive chemical group via the cysteine at position 128 of the heavy chain variable domain according to the IMGT nomenclature of the mutant antibody fragment.

20. The intermediate conjugate of claim 19 having the formula R¹—X¹-L¹-Z wherein R¹, X¹ and L¹ are as defined in claim 14, and Z represents a reactive chemical group selected from 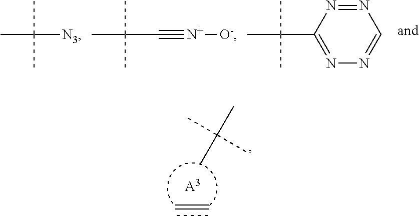
with ring A³ representing a cycloalkyne, cycloalkene, heterocycloalkyne or heterocycloalkene ring, optionally fused with one or more aromatic rings and optionally substituted with one or more groups selected from (C1-C6)alkyl, halide, and hydroxy.

21. (canceled)

22. (canceled)
